(19) United States
(12) Patent Application Publication
(10) Pub. No.: US 2002/0137746 A1
(43) Pub. Date: Sep. 26, 2002

(54) Method of treating or preventing urinary incontinence using prostanoid EP1 receptor antagonists

(76) Inventors: Francois Joseph Carl, Kirkland (CA); Kathleen Mary Metters, Montreal (CA); Theodore Paul Broten, Ambler, PA (US); Mervyn Tuner, Westfield, NJ (US)

Correspondence Address: MERCK AND CO INC, P O BOX 2000, RAHWAY, NJ, 070650907

(21) Appl. No.: 09/935,614

(22) Filed: Aug. 23, 2001

Related U.S. Application Data
(60) Provisional application No. 60/227,183, filed on Aug. 23, 2000.

(51) Int. Cl. A61K 31/53; A61K 31/506; A61K 31/501; A61K 31/497; A61K 31/4436; A61K 31/4245

(52) U.S. Cl. 514/241; 514/252.03; 514/252.01; 514/255.05; 514/256; 514/340; 514/342; 514/397; 514/422; 514/372; 514/374; 514/364; 514/378; 514/406

(57) ABSTRACT

This invention encompasses a method of treating or preventing urinary incontinence in a mammalian patient comprising administering to the patient a compound of formula I:

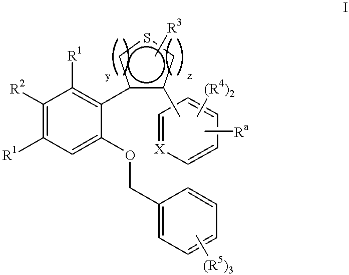

or a pharmaceutically acceptable salt, hydrate or ester thereof. The invention also encompasses certain pharmaceutical compositions and methods for treatment of prostaglandin mediated diseases comprising the use of compounds of formula I.

### BACKGROUND OF THE INVENTION

The present invention relates to the use of prostanoid EP1 receptor antagonist compounds for treating or preventing urinary incontinence. Urinary incontinence as used herein refers to the inability to retain urine in the bladder. It results from neurologic or mechanical disorders of the complicated system that controls normal micturition. Detrusor instability, also known as urge urinary incontinence causes at least fifty percent of urinary incontinence among the elderly. The usual clinical picture is of unpredictable, involuntary voiding. This pathology clearly has a detrimental effect on daily life and causes the afflicted persons to avoid social contacts.

Prostanoids derived via the cyclooxygenase pathway have been demonstrated to affect the micturition response through effects on bladder activity. Prostanoids may act as endogenous modulators of the afferent input from the bladder to the CNS. Prostaglandins may also affect bladder activity by modulation of the smooth muscle contractility response of the bladder. The afferent branch of the micturition reflex is a major target for prostanoid action as local modulators of micturition. This is supported by cystometric experiments in which rats were given cyclooxygenase inhibitors. Cyclooxygenase inhibitors exert a mild depressant effect of electrically evoked, nerve mediated contractions of the mammalian detrusor. Moreover, the addition of exogenous prostaglandins increases urinary bladder smooth muscle contractility both through direct action on the smooth muscle and through increased nerve-mediated contractions. These data suggest that blockade or antagonism of the actions of prostaglandins on the prostaglandin receptors involved in micturition response may be useful for the treatment or prevention of urinary incontinence.

Five prostanoids responsible for the majority of bioactivities include: prostaglandin E2, prostaglandin D2, prostaglandin F2α, prostacyclin and thromboxane A2. These prostanoids are known to activate a family of at least eight membrane-bound receptors which have been identified as EP1, EP2, EP3 and EP4 for prostaglandin E2, DP for prostaglandin D2, FP for prostaglandin F2α, IP for prostacyclin and TP for thromboxane A2.

Consequently one object of the invention is to provide a new treatment modality for urinary incontinence in mammalian patients.

Another object of the present invention is to provide a treatment for urinary incontinence that utilizes a mechanism of action which is distinctly different from that employed by anti-muscarinics.

These and other objects of the invention will be apparent to those of ordinary skill from the teachings herein.

### BRIEF DESCRIPTION OF THE DRAWINGS

The invention is described herein in connection with the following drawings in which:

FIG. 1 is a graph showing micturition events per minute in laboratory animals, and

FIG. 2 is a graph showing the micturition interval in laboratory animals.

### SUMMARY OF THE INVENTION

A method of treating or preventing urinary incontinence is disclosed comprising administering to a mammalian patient in need of such treatment or prevention a compound represented by formula I:

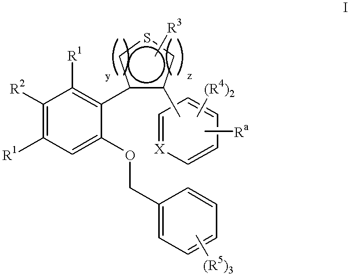

or a pharmaceutically acceptable salt, hydrate or ester thereof, wherein:

X represents C or N;

y and z are independently 0-2, such that y+z=2;

R^{a} is selected from the group consisting of:

1) heteroaryl, wherein heteroaryl is selected from the group consisting of:

a) furyl,

b) diazinyl, triazinyl or tetrazinyl,

c) imidazolyl,

d) isoxazolyl,

e) isothiazolyl,

f) oxadiazolyl,

g) oxazolyl,

h) pyrazolyl,

i) pyrrolyl,

j) thiadiazolyl,

k) thiazolyl

l) thienyl

m) triazolyl and

n) tetrazolyl,

said heteroaryl group being optionally substituted with one to three substituents selected from R¹¹ and C₁₋₄alkyl,

2) —COR⁶,

3) —NR⁷R⁸,

4) —SO₂R⁹,

5) hydroxy,

6) C₁₋₆alkoxy, optionally substituted with one to three substituents selected from R¹¹, and

7) C₁₋₆alkyl, C₂₋₆alkenyl or C₃₋₆cycloalkyl, optionally substituted with one to three substituents selected from R¹¹, and further substituted with 1-3 substituents selected from the group consisting of:

(a) —COR⁶

(b) —NR⁷R⁸,

(c) —SO₂R⁹,

(d) hydroxy,

(e) C₁₋₆alkoxy or halo C₁₋₆alkoxy, and

(f) heteroaryl,

such that R^{a} is positioned on the phenyl ring to which it is bonded in a 1,3 or 1,4 relationship relative to the thienyl group represented in formula I;

each R¹, R², R³, R⁴ and R⁵ are independently selected from the group consisting of:

1) hydrogen,

2) halogen,

3) C₁₋₆alkyl,

4) C₁₋₆alkoxy,

5) C₁₋₆alkylthio,

6) nitro,

7) carboxy and

8) CN, wherein items (3)-(5) above are optionally substituted with one or more substituents independently selected from R¹¹;

R⁶ is selected from the group consisting of hydrogen, hydroxy, C₁₋₆alkyl, C₁₋₆alkoxy and NR⁷R⁸, wherein C₁₋₆alkyl or C₁₋₆alkoxy are optionally substituted with one or more substituents independently selected from R¹¹;

R⁷ and R⁸ are independently selected from the group consisting of:

(1) hydrogen,

(2) hydroxy,

(3) SO₂R⁹

(4) C₁₋₆alkyl,

(5) C₁₋₆alkoxy,

(6) phenyl,

(7) naphthyl,

(8) furyl,

(9) thienyl and

(10) pyridyl, wherein items (4)-(5) above are optionally substituted with one or more substituents independently selected from R¹¹, and items (6)-(10) above are optionally substituted with one or more substituents independently selected from R¹¹ or C₁₋₄alkyl,

R⁹ is selected from the group consisting of

(1) hydroxy,

(2) N(R¹⁰)₂,

(3) C₁₋₆alkyl, optionally substituted with one or more substituents independently selected from R¹¹,

(4) phenyl,

(5) naphthyl,

(6) furyl,

(7) thienyl and

(8) pyridyl, wherein items (4)-(8) above are optionally substituted with one or more substituents independently selected from R¹¹ or C₁₋₄alkyl;

R¹⁰ is hydrogen or C₁₋₆alkyl; and

R¹¹ is the group consisting of halogen, hydroxy, C₁₋₃alkoxy, nitro, N(R¹⁰)₂ and pyridyl.

### DETAILED DESCRIPTION OF THE INVENTION

The invention is described using the following definitions unless otherwise indicated.

The term “halogen” or “halo” includes F, Cl, Br, and I.

The term “alkyl” means linear, branched or cyclic structures and combinations thereof, having the indicated number of carbon atoms. Thus, for example, C₁₋₆alkyl includes methyl, ethyl, propyl, 2-propyl, s- and t-butyl, butyl, pentyl, hexyl, 1,1-dimethylethyl, cyclopropyl, cyclobutyl, cyclopentyl and cyclohexyl.

“Alkoxy” means alkoxy groups of a straight, branched or cyclic configuration having the indicated number of carbon atoms. C₁₋₆alkoxy, for example, includes methoxy, ethoxy, propoxy, isopropoxy, and the like.

“Alkylthio” means alkylthio groups having the indicated number of carbon atoms of a straight, branched or cyclic configuration. C₁₋₆alkylthio, for example, includes methylthio, propylthio, isopropylthio, etc.

“Haloalkyl” means an alkyl group as described above wherein one or more hydrogen atoms have been replaced by halogen atoms, with up to complete substitution of all hydrogen atoms with halo groups. C₁₋₆haloalkyl, for example, includes —CF₃, —CF₂CF₃ and the like.

“Haloalkoxy” means an alkoxy group as described above in which one or more hydrogen atoms have been replaced by halogen atoms, with up to complete substitution of all hydrogen atoms with halo groups. C₁₋₆haloalkoxy, for example, includes —OCF₃, —OCF₂CF₃ and the like.

“Alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

For purposes of this specification, the following abbreviations have the indicated meanings:

| BOC | = t-butyloxycarbonyl | Me | = methyl |
|---|---|---|---|
| CBZ | = carbobenzoxy | Et | = ethyl |
| DCC | = 1,3-dicyclohexylcarbodiimide | n-Pr | = normal propyl |
| DIBAL | = diisobutyl aluminum hydride | i-Pr | = isopropyl |
| DIEA | = N,N-diisoproylethylamine | n-Bu | = normal butyl |
| DMAP | = 4-(dimethylamino)pyridine | i-Bu | = isobutyl |
| EDCI | = 1-(3-dimethylaniinopropyl)-3-ethylcarbodiimide | | |
| | hydrochloride | | |
| EDTA | = ethlenediaminetetraacetic acid, tetrasodium salt hydrate | | |
| FAB | = fast atom bombardment | s-Bu | = secondary butyl |
| FMOC | = 9-fluorenylmethoxycarbonyl | t-Bu | = tertiary butyl |

| | |
|---|---|
| HMPA | = hexamethylphosphoramide |
| HATU | = O-(7-Azabenzotriazol-1-yl)N,N,N’,N’-tetramethyluronium |
| | hexafluorophosphate |
| HOBt | = 1-hydroxybenzotriazole |
| HRMS | = high resolution mass spectrometry |
| ICBF | = isobutyl chioroformate |
| NBS | = N-bromosuccinimide |
| MsO | = methanefulfonate =mesylate |
| Ms | = methanesulfonyl =mesyl |
| MCPBA | = metachloroperbenzoic acid |
| LDA | = lithium diisopropylamide |
| KHMDS | = potassium hexamethyldisilazane |
| NMM | = 4-methylmorpholine |
| PCC | = pyridinium chlorochromate |
| PDC | = pyridinium dichromate |
| Ph | = phenyl |
| PPTS | = pyridinium p-toluene sulfonate |
| pTSA | = p-toluene sulfonic acid |
| r.t. | = room temperature |
| rac. | = racemic |
| TfO | = trifluoromethanesulfonate = triflate |
| TLC | = thin layer chromatography |

In one aspect of the invention, the invention relates to a method of treating or preventing urinary incontinence comprising administering to a mammalian patient in need such treatment or prevention a compound represented by formula I:

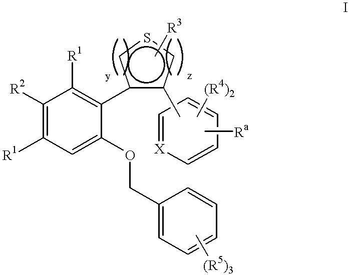

or a pharmaceutically acceptable salt, hydrate or ester thereof, wherein:

X represents C or N;

y and z are independently 0-2, such that y+z=2;

R^{a} is selected from the group consisting of:

1) heteroaryl, wherein heteroaryl is selected from the group consisting of:

a) furyl,

b) diazinyl, triazinyl or tetrazinyl,

c) imidazolyl,

d) isoxazolyl,

e) isothiazolyl,

f) oxadiazolyl,

g) oxazolyl,

h) pyrazolyl,

i) pyrroyl,

j) thiadiazloyl,

k) thiazolyl

l) thienyl

m) triazolyl and

n) tetrazolyl, wherein heteroaryl is optionally substituted with 1-3 substituents independently selected from R¹¹ or C₁₋₄alkyl,

2) —COR⁶,

3) —NR⁷R⁸,

4) —SO₂R⁹,

5) hydroxy,

6) C₁₋₆alkoxy, optionally substituted with 1-3 substituents independently selected from R¹¹, and

7) C₁₋₆alkyl, C₂₋₆alkenyl or C₃₋₆cycloalkyl, optionally substituted with 1-3 substituents independently selected from R¹¹, and further substituted with 1-3 substituents independently selected from the group consisting of:

(a) —COR⁶

(b) —NR⁷R⁸,

(c) —SO₂R⁹,

(d) hydroxy,

(e) C₁₋₆alkoxy or halo C₁₋₆alkoxy, and

(f) heteroaryl,

such that R^{a} is positioned on the pyridyl ring to which it is bonded in a 1,3 or 1,4 relationship relative to the thienyl group represented in formula I;

R¹, R², R³, R⁴ and R⁵ are independently selected from the group consisting of:

1) hydrogen,

2) halogen,

3) C₁₋₆alkyl,

4) C₁₋₆alkoxy,

5) C₁₋₆alkylthio,

6) nitro,

7) carboxy and

8) CN, wherein items (3)-(5) above are optionally substituted with 1-3 substituents independently selected from R¹¹;

R⁶ is selected from the group consisting of hydrogen, hydroxy, C₁₋₆alkyl, C₁₋₆alkoxy and NR⁷R⁸, wherein C₁₋₆alkyl or C₁₋₆alkoxy are optionally substituted with 1-3 substituents independently selected from R¹¹;

R⁷ and R⁸ are independently selected from the group consisting of:

(1) hydrogen,

(2) hydroxy,

(3) SO₂R⁹

(4) C₁₋₆alkyl,

(5) C₁₋₆alkoxy,

(6) phenyl,

(7) naphthyl,

(8) furyl,

(9) thienyl and

(10) pyridyl, wherein items (4)-(5) above are optionally substituted with 1-3 substituents independently selected from R¹¹, and items (6)-(10) above are optionally substituted with 1-3 substituents independently selected from RI 1 or C₁₋₄alkyl,

R⁹ is selected from the group consisting of

(1) hydroxy,

(2) N(R¹⁰)₂,

(3) C₁₋₆alkyl, optionally substituted with 1-3 substituents independently selected from R¹¹,

(4) phenyl,

(5) naphthyl,

(6) furyl,

(7) thienyl and

(8) pyridyl, wherein items (4)-(8) above are optionally substituted with 1-3 substituents independently selected from R¹¹ or C₁₋₄alkyl;

R¹⁰ is hydrogen or C₁₋₆alkyl; and

R¹¹ is selected from the group consisting of: halogen, hydroxy, C₁₋₃alkoxy, nitro, N(R¹⁰)₂ and pyridyl.

An embodiment of the present invention that is of particular interest relates to a method using a compound of formula I wherein X represents C. Within this subset, all other variables are as originally defined.

Another embodiment of the present invention that is of particular interest relates to the use of a compound of formula I wherein X represents N. Within this subset, all other variables are as originally defined.

Another embodiment of the invention that is of particular interest relates to the use of a compound of formula I wherein R^{a} is selected from the group consisting of: heteroaryl, which is as originally defined, COR⁶, wherein R⁶ is as originally defined, C₁₋₆ alkyl and C₂₋₆ alkenyl, optionally substituted as originally defined, and SO₂R⁹ with R⁹ as originally defined. Within this subset, all other variables are as originally defined.

More particularly, an embodiment of the invention that is of interest relates to the use of a compound of formula I wherein R^{a} is selected from the group consisting of:

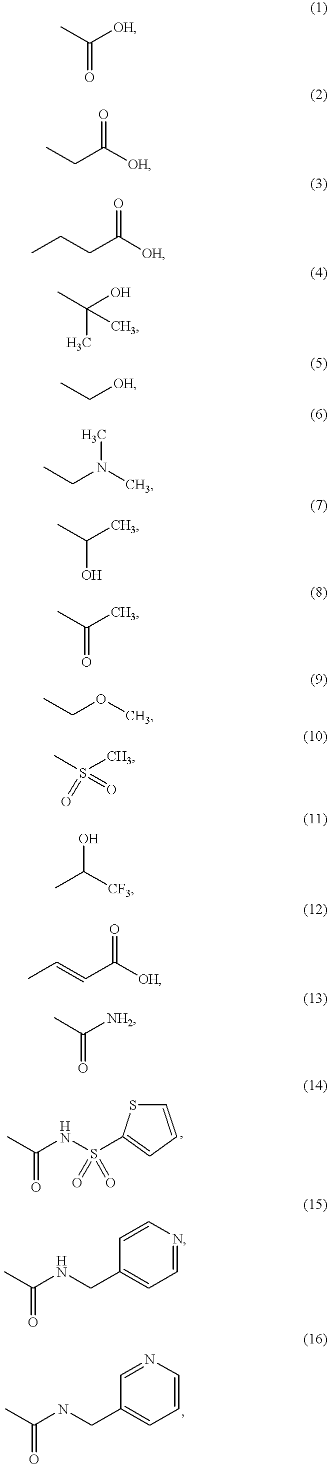

Within this embodiment of the invention, all other variables are as originally defined.

More particularly, a subset of the invention that is of interest relates to the use of a compound of formula I wherein R^{a} is selected from the group consisting of: CO₂H, CH₂OH, C(OH)(CH₃)₂, CH(OH)CF₃ and C(O)CF₃. Within this subset, all other variables are as originally defined.

Another embodiment of the invention that is of particular interest relates to the use of a compound of formula I wherein 1-3 of R¹ and R² are selected from the group consisting of halo, C₁₋₄ alkyl, C₁₋₄ alkoxy, C₁₋₄ alkylthio and NO₂. Within this subset, all other variables are as originally defined.

Another embodiment of the invention that is of particular interest relates to the use of a compound of formula I wherein each R⁴ and R⁵ independently represents a member selected from the group consisting of: H, halo, C₁₋₆ alkyl and C₁₋₆ alkoxy, said alkyl and alkoxy groups being optionally substituted as originally defined. Within this subset, all other variables are as originally defined.

Another embodiment of the invention that is of particular interest relates to the use of a compound of formula I wherein each R³ independently represents a member selected from the group consisting of: H and halo. Within this subset, all other variables are as originally defined.

Another embodiment of the invention that is of particular interest relates to the use of a compound of formula I wherein one of y and z represents 0 and the other represents 2. Within this subset, all other variables are as originally defined.

An embodiment of the invention that is of more particular interest relates to the use of a compound of formula I wherein X is C or N;

R^{a} is selected from the group consisting of: heteroaryl, as originally defined, COR⁶, wherein R⁶ is as originally defined, C₁₋₆ alkyl and C₂₋₆ alkenyl, optionally substituted as originally defined, and SO₂R⁹ with R⁹ as originally defined;

R¹ and R² are selected from the group consisting of halo, C₁₋₄ alkyl, C₁₋₄ alkoxy, C₁₋₄ alkylthio and NO₂;

R⁴ and R⁵ independently represent members selected from the group consisting of: H, halo, C₁₋₆ alkyl and C₁₋₆ alkoxy, said alkyl and alkoxy groups being optionally substituted as originally defined;

each R³ independently represents a member selected from the group consisting of: H and halo;

and one of y and z represents 0 and the other represents 2. Within this subset, all other variables are as originally defined.

Another embodiment of the invention that is of more particular interest relates to the use of a compound of formula I wherein:

X is C or N;

R^{a} is selected from the group consisting of:

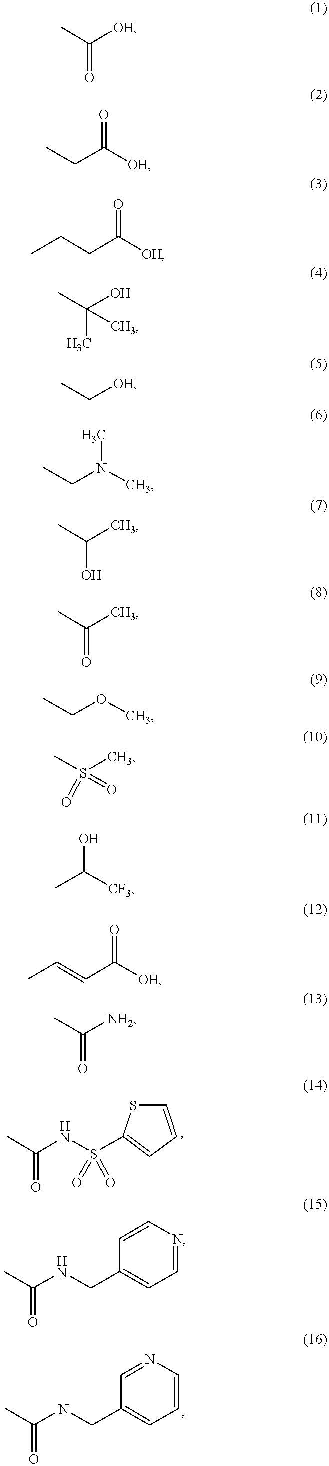

R¹ and R² are selected from the group consisting of halo, C₁₋₄ alkyl, C₁₋₄ alkoxy, C₁₋₄ alkylthio and NO₂;

R⁴ and R⁵ independently represent members selected from the group consisting of: H, halo, C₁₋₆ alkyl and C₁₋₆ alkoxy, said alkyl and alkoxy groups being optionally substituted as originally defined;

each R³ independently represents a member selected from the group consisting of: H and halo;

and one of y and z represents 0 and the other represents 2. Within this subset, all other variables are as originally defined.

Another embodiment of the invention that is of more particular interest relates to the use of a compound of formula I wherein:

X is C or N;

R^{a} is selected from the group consisting of: CO₂H, CH₂OH, C(OH)(CH₃)₂, CH(OH)CF₃ and C(O)CF₃;

R¹ and R² are selected from the group consisting of halo, C₁₋₄ alkyl, C₁₋₄ alkoxy, C₁₋₄ alkylthio and NO₂;

R⁴ and R⁵ independently represent members selected from the group consisting of: H, halo, C₁₋₆ alkyl and C₁₋₆ alkoxy, said alkyl and alkoxy groups being optionally substituted as originally defined;

each R³ independently represents a member selected from the group consisting of: H and halo;

and one of y and z represents 0 and the other represents 2. Within this subset, all other variables are as originally defined.

Examples of compounds that are useful in the method described herein are found in table I below.

TABLE 1

| COMPOUND | EXAMPLE |
|---|---|
| 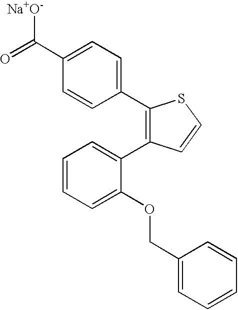 | 1 |
| 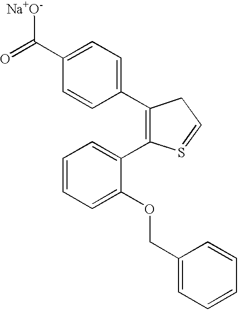 | 2 |
| 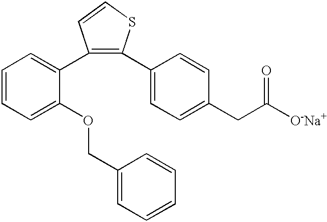 | 3 |
| 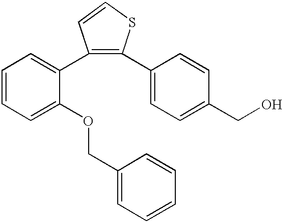 | 4 |
| 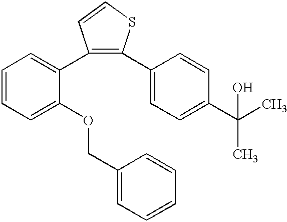 | 5 |
| 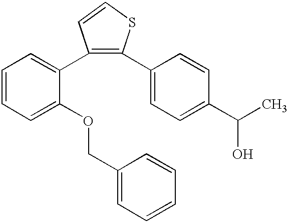 | 6 |
| 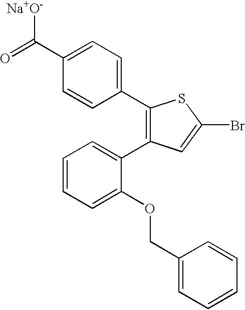 | 7 |
| 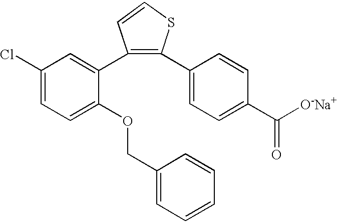 | 8 |
| 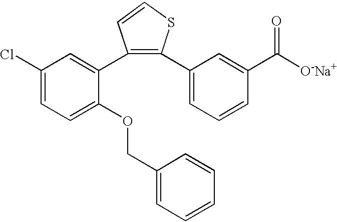 | 9 |
| 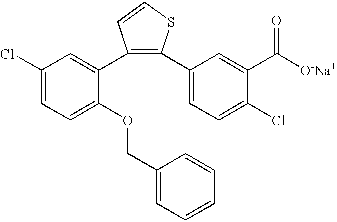 | 10 |
| 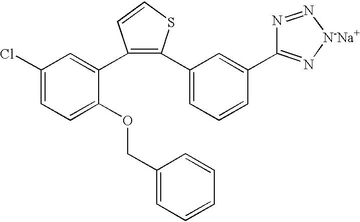 | 11 |
| 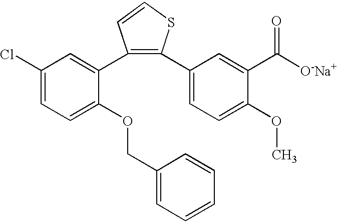 | 12 |
| 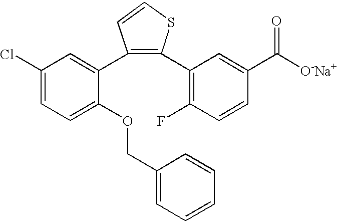 | 13 |
| 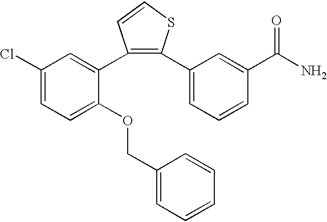 | 14 |
| 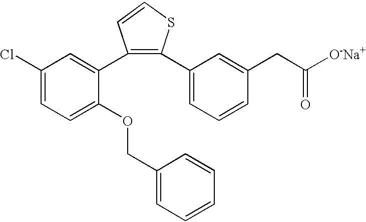 | 15 |
| 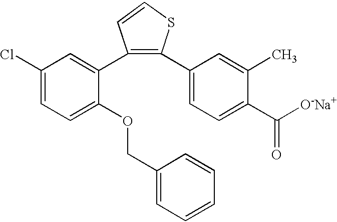 | 16 |
| 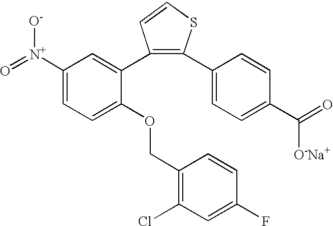 | 17 |
| 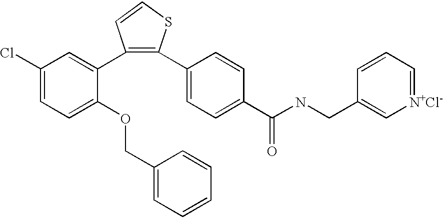 | 18 |
| 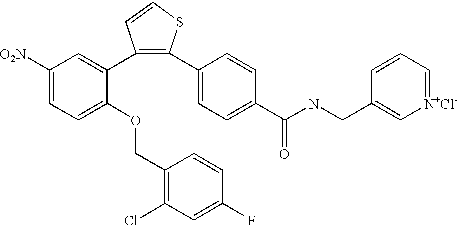 | 19 |
| 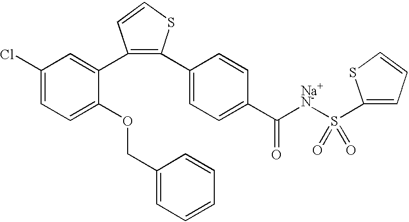 | 20 |
| 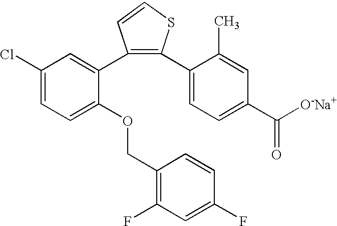 | 21 |
| 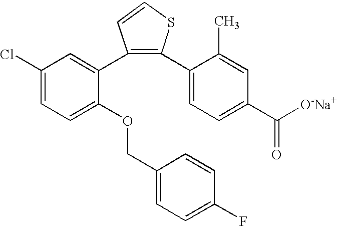 | 22 |
| 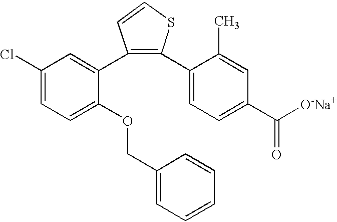 | 23 |
| 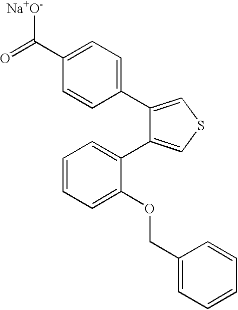 | 25 |
| 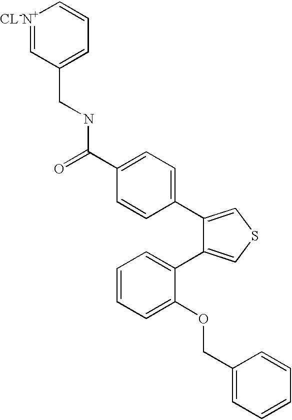 | 26 |
| 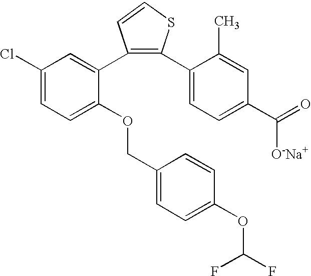 | 27 |
| 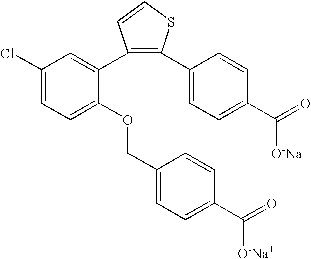 | 28 |
| 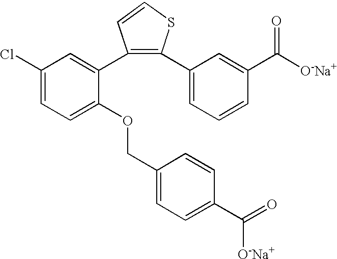 | 29 |
| 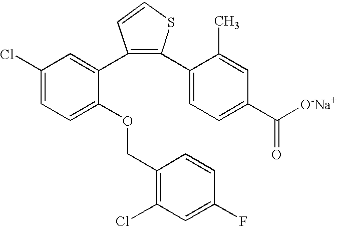 | 30 |
| 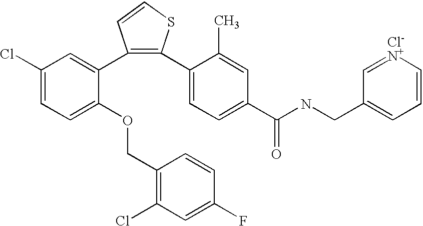 | 31 |
| 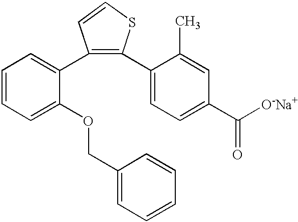 | 37 |
| 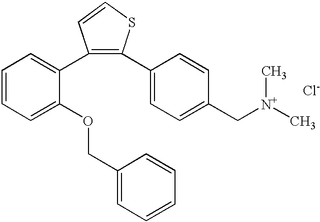 | 38 |
| 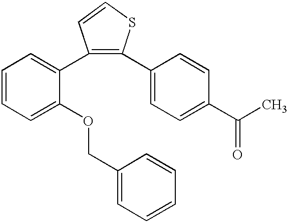 | 39 |
| 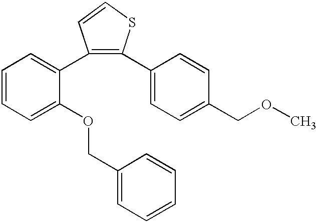 | 40 |
| 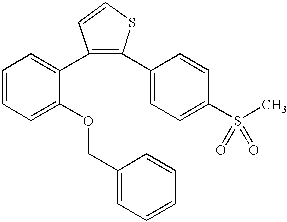 | 41 |
| 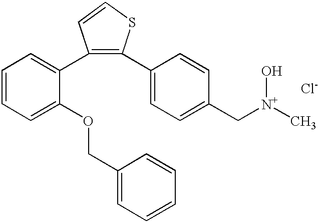 | 43 |
| 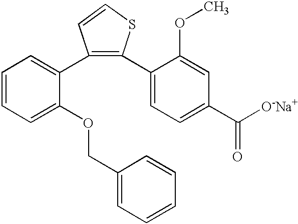 | 44 |
| 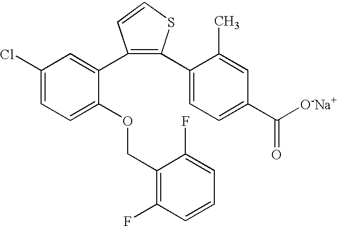 | 45 |
| 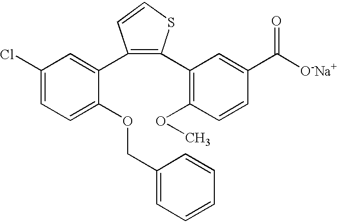 | 46 |
| 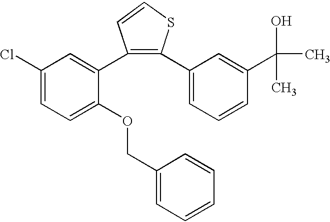 | 47 |
| 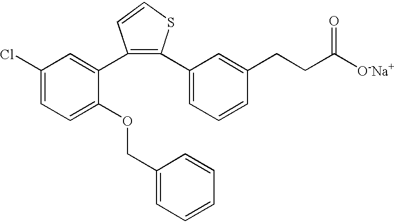 | 48 |
| 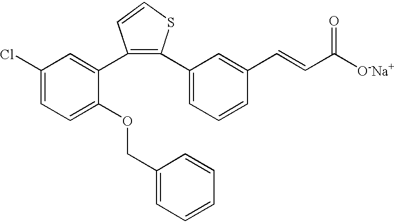 | 49 |
| 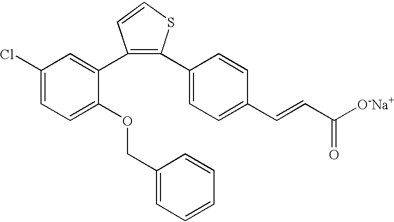 | 50 |
| 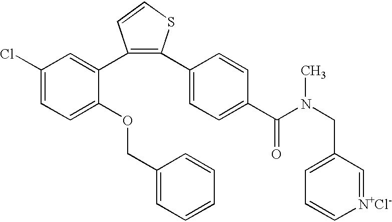 | 52 |
| 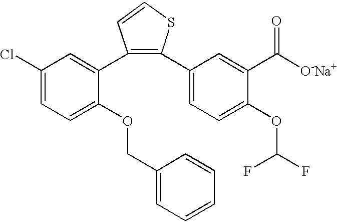 | 53 |
| 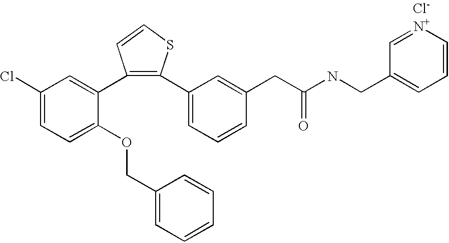 | 55 |
| 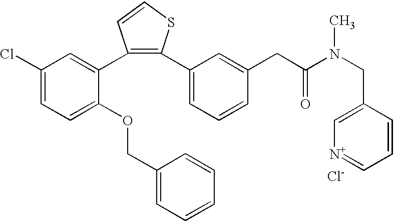 | 56 |
| 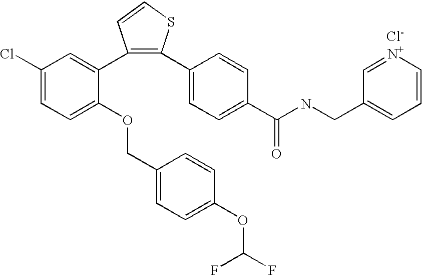 | 57 |
| 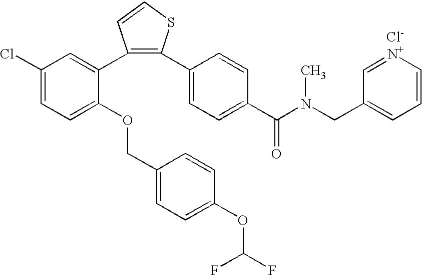 | 58 |
| 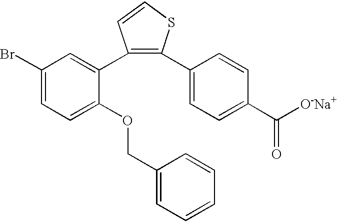 | 59 |
| 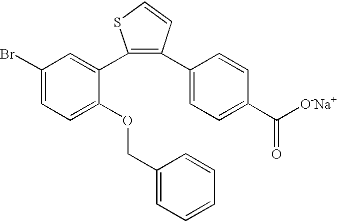 | 60 |
| 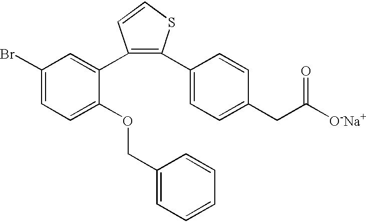 | 61 |
| 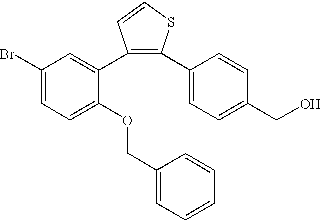 | 62 |
| 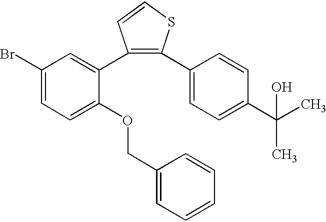 | 63 |
| 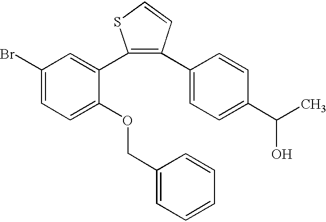 | 64 |
| 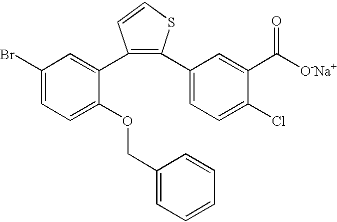 | 65 |
| 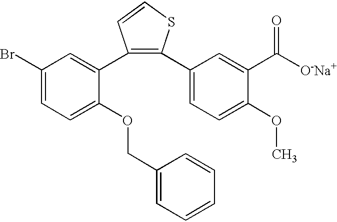 | 66 |
| 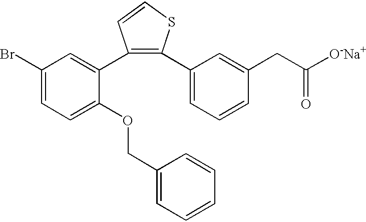 | 67 |
| 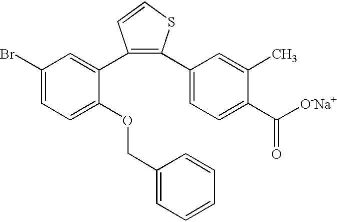 | 68 |
| 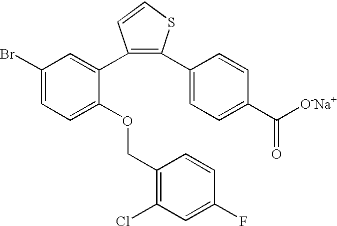 | 69 |
| 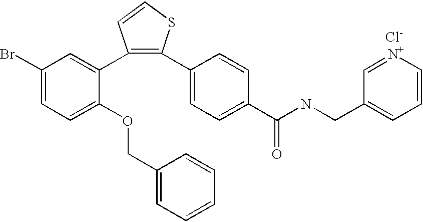 | 70 |
| 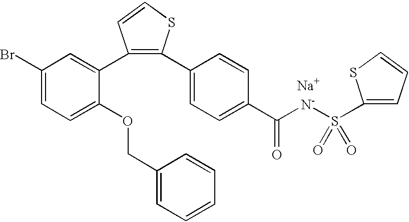 | 71 |
| 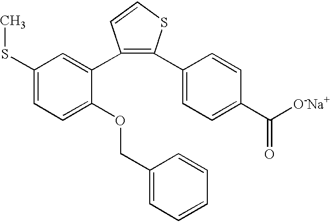 | 76 |
| 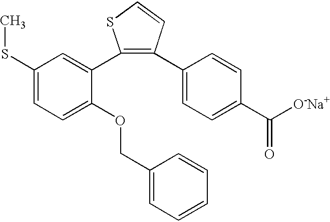 | 77 |
| 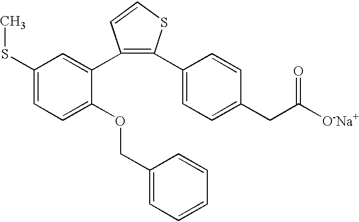 | 78 |
| 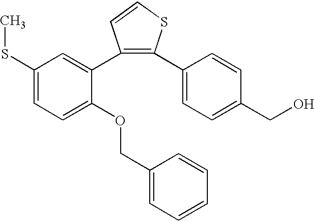 | 79 |
| 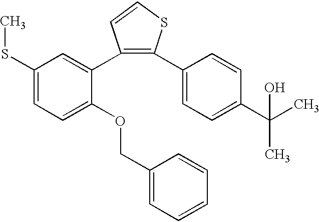 | 80 |
| 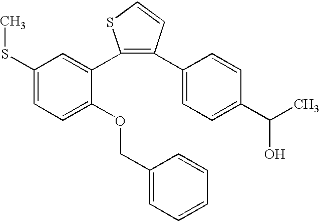 | 81 |
| 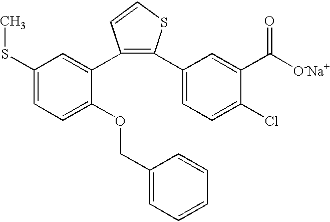 | 82 |
| 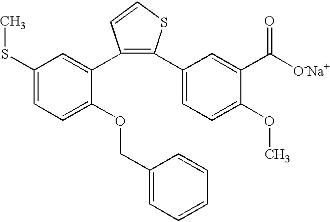 | 83 |
| 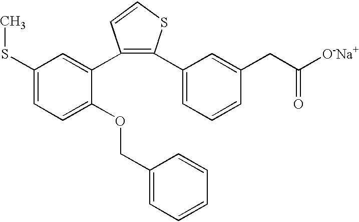 | 84 |
| 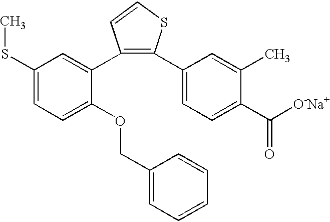 | 85 |
| 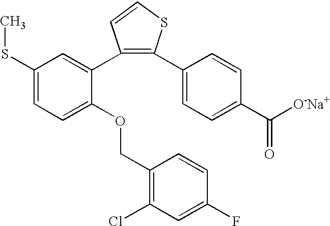 | 86 |
| 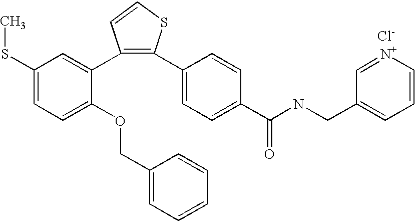 | 87 |
| 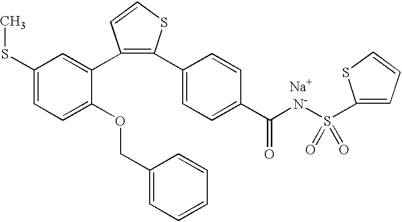 | 88 |
| 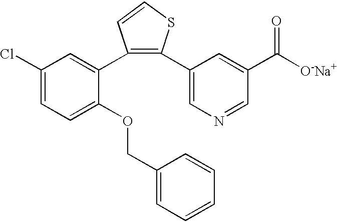 | 1a |
| 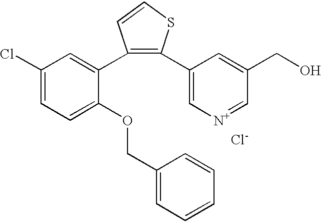 | 2a |
| 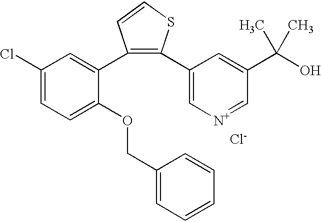 | 3a |
| 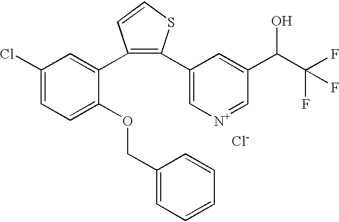 | 4a |
| 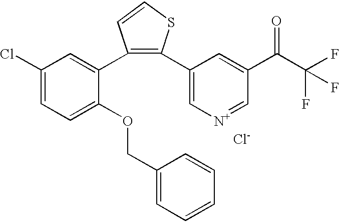 | 5a |
| 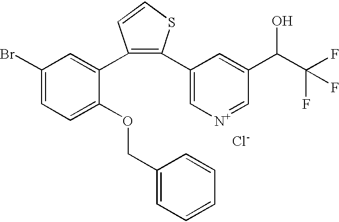 | 6a |
| 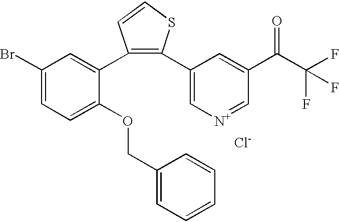 | 7a |

Another embodiment of the invention is a method of treating or preventing urinary incontinence comprising administering to a mammalian patient in need of such treatment a compound of formula I in an amount that is effective for treating or preventing urinary incontinence, wherein the compound is co-administered with one or more other agents or ingredients.

Another embodiment of the invention is a method of treating or preventing urinary incontinence comprising administering to a mammalian patient in need of such treatment a compound of formula I in an amount which is effective for treating or preventing urinary incontinence, wherein the compound is co-administered with another agent or ingredient selected from the group consisting of: a cyclooxygenase-2 selective inhibiting drug, such as rofecoxib, celecoxib, etoricoxib and the like, a diuretic, such as chlorothiazide, hydrochlorothiazide, furosemide and the like, a potassium supplement, such as potassium chloride, potassium gluconate and the like, potassium channel modulators, such as pinacidil, cromakalin and the like, a urinary antiseptic, such as nitrofurantoin, methenamine mandelate or hipprate and the like, a muscle relaxant, such as flavoxate, an alpha adrenoceptor antagonist, such as prazosin, doxazosin and the like, a beta adrenoceptor agonist, such as clenbuterol, salbutamol and the like, an anticholinergic, such as atropine, scopolamine, ditropan and the like, calcium channel antagonists, such as nifedipine, vasopressin analogues such as desmopressin and the like, antimuscarinics, such as tolterodine, emepronium, oxybutynin and the like, estrogen replacements, such as conjugated estrogens, neurokinin receptor antagonists, dopamine receptor ligands, phosphodiesterase inhibitors such as vinpocetine and rolipram, an antidepressant, such as imipramine, and other agents, such as baclofen, resineferatoxin and capsaicin.

The magnitude of a prophylactic or therapeutic dose of a compound of formula I will, of course, vary with the nature and the severity of the condition to be treated and with the particular compound of formula I and its route of administration. It will also vary according to a variety of factors including the age, weight, general health, sex, diet, time of administration, rate of excretion, drug combination and response of the individual patient. In general, the daily dose from about 0.001 mg to about 100 mg per kg body weight of a mammal, preferably about 0.01 mg to about 10 mg per kg. On the other hand, it may be necessary to use dosages outside these limits in some cases.

The amount of active ingredient that may be combined with the carrier materials to produce a single dosage form will vary depending upon the host treated and the particular mode of administration. For example, a formulation intended for the oral administration of humans may contain from about 0.5 mg to 5 g of active agent compounded with an appropriate and convenient amount of carrier material which may vary from about 5 to about 95 percent of the total composition. Dosage unit forms will generally contain between from about 1 mg to about 2 g of an active ingredient, typically 25 mg, 50 mg, 100 mg, 200 mg, 300 mg, 400 mg, 500 mg, 600 mg, 800 mg or 1000 mg.

For the treatment of urinary incontinence, the compound of formula I may be administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, cattle, sheep, dogs, cats, etc., the compound of the invention is effective in the treatment of humans.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tracts and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin or olive oil.

Aqueous suspensions contain the active material in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of formula I may also be administered in the form of suppositories for rectal administration of the drug. These compositions can be prepared by mixing the drug with a suitable non-irritating excipient which is solid at ambient temperatures but liquid at the rectal temperature and will therefore melt in the rectum to release the drug. Such materials are cocoa butter and polyethylene glycols.

For topical use, creams, ointments, gels, solutions or suspensions, etc., containing the compound of formula I are employed. (For purposes of this application, topical application shall include mouth washes and gargles.) Topical formulations may generally be comprised of a pharmaceutical carrier, cosolvent, emulsifier, penetration enhancer, preservative system, and emollient.

The ability of the compounds of formula I to interact with prostaglandin receptors makes them useful for preventing or reversing undesirable symptoms caused by prostaglandins in a mammalian, especially human subject. This mimicking or antagonism of the actions of prostaglandins indicates that the compounds and pharmaceutical compositions thereof are useful to treat, prevent, or ameliorate the signs and symptoms of urinary incontinence and related bladder disorders in mammals and especially in humans. Compounds of formula I will also inhibit prostanoid-induced smooth muscle contraction by antagonizing contractile prostanoids or mimicking relaxing prostanoids and hence may be use in the treatment or prevention described herein.

Methods of Synthesis

Compounds of the present invention can be prepared according to the following methods.

Preparation of Common Intermediates

As illustrated in Scheme 1, the thiophene derivative (3) is prepared by reacting 2-bromophenylbenzyl ether (1) with thiophene 3-boronic acid (2) under conditions such as palladium catalyzed Suzuki's cross coupling reaction. The thiophene derivative (3) can then be brominated selectively at the 2 position using reagents such as NBS in THF/H₂O (50:1). Subsequent Suzuki's cross coupling reaction with the desired boronic acid (5) affords the desired benzoic acid derivative (6).

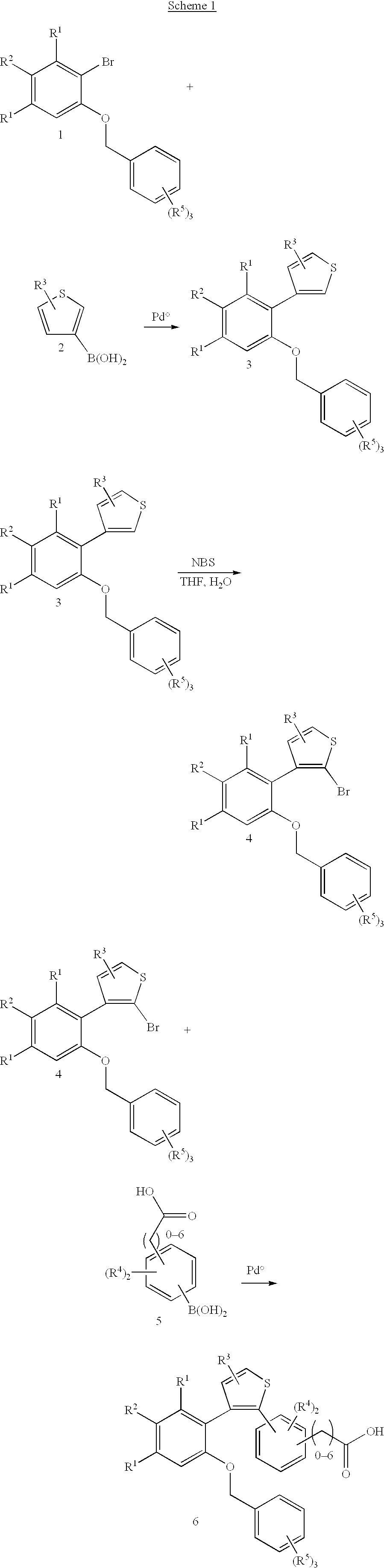

In a similar fashion, the thiophene isomers (13) can be prepared as shown in Scheme 2 by submitting 3-bromothiophene compounds (7) under palladium catalyzd Suzuki's cross coupling with the desired boronic acid (8) to give the derivative (9), which is then brominated selectively at the 2 position using reagents such as NBS in THF/H₂O (50:1). Finally, palladium catalyzed Suzuki's cross coupling reaction between the bromo derivative (10) and the desired boronic acid (11) followed by basic hydrolysis of the ester lead to the desired product (13).

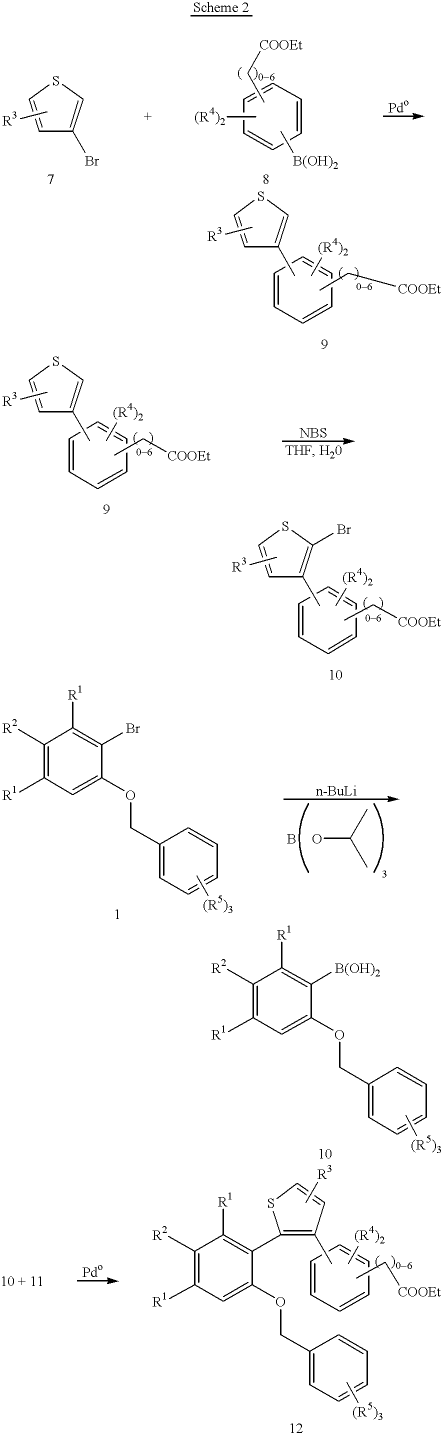

As illustrated in Scheme 2a, compound 20 can be prepared via a palladium catalyzed Stille cross coupling reaction of the bromo derivative (19) and stannane (18) which is prepared with hexamethylditin.

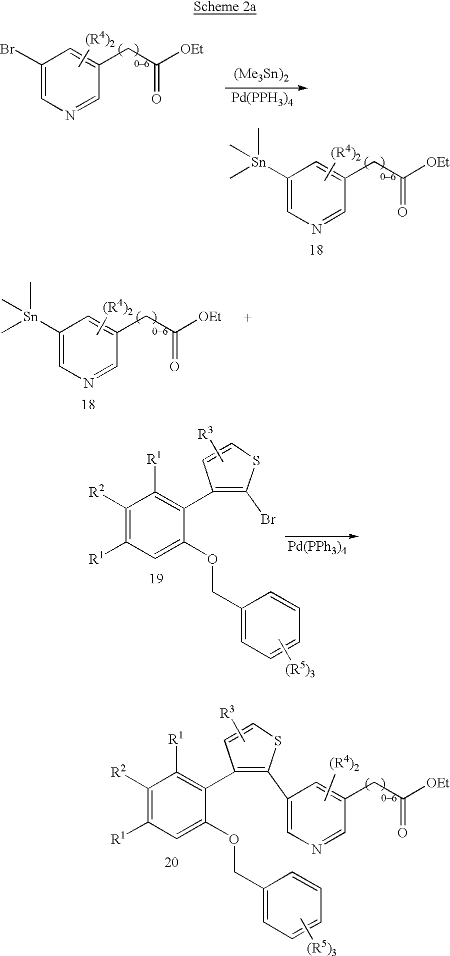

Compound 20 can then be hydrolyzed under basic conditions to give the corresponding acid.

The other thiophene isomer (17) is prepared via a multi-step sequence as shown in Scheme 3,3,4-dibromothiophene (14) is reacted with the boronic acid (8) to yield the derivative (15), which is once submitted to palladium catalyzed Suzuki's cross coupling reaction with the desired boronic acid (11) followed by basic hydrolysis to afford the desired material (17).

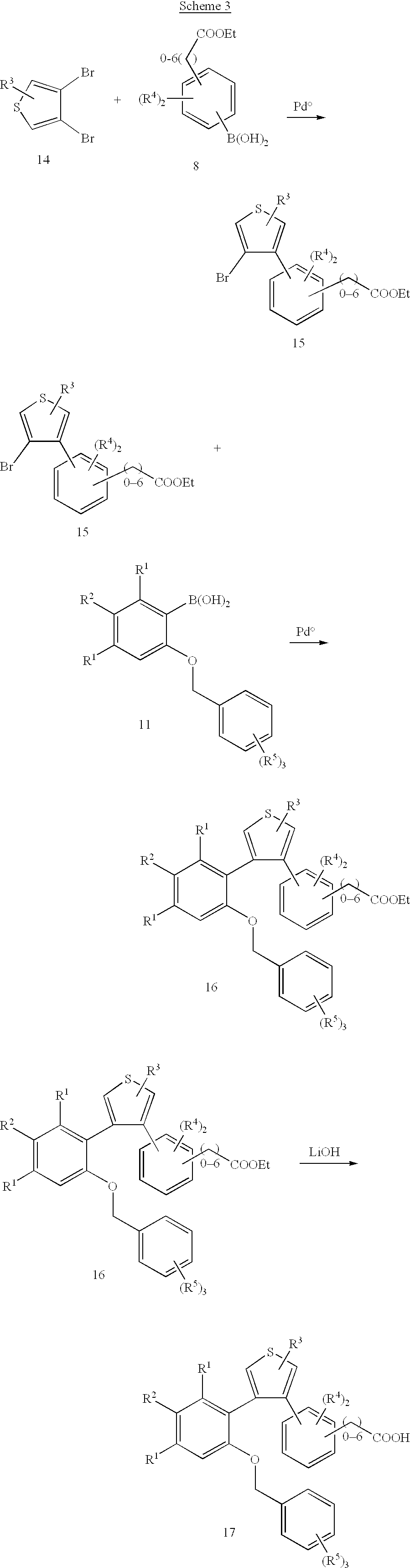

Position isomers of compound 20 can be made according to Schemes 3a and 4a as follows:

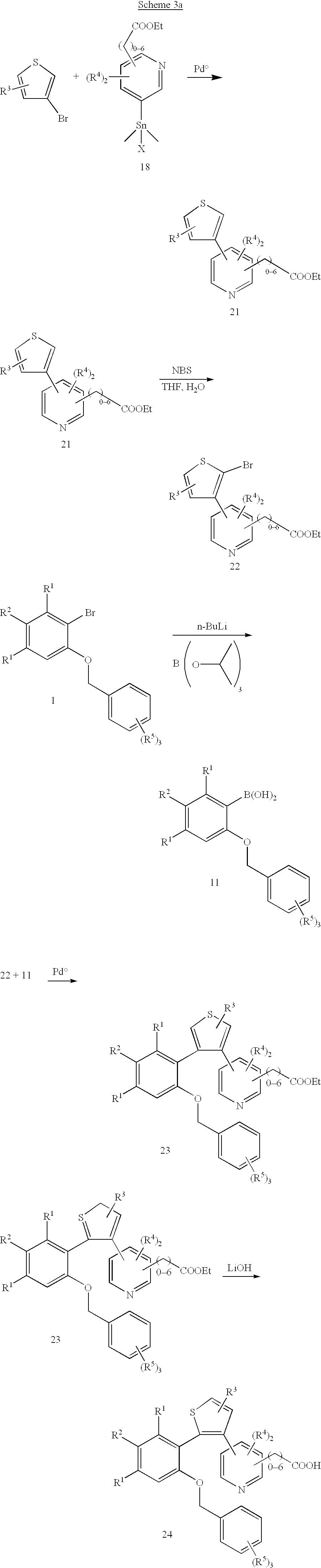

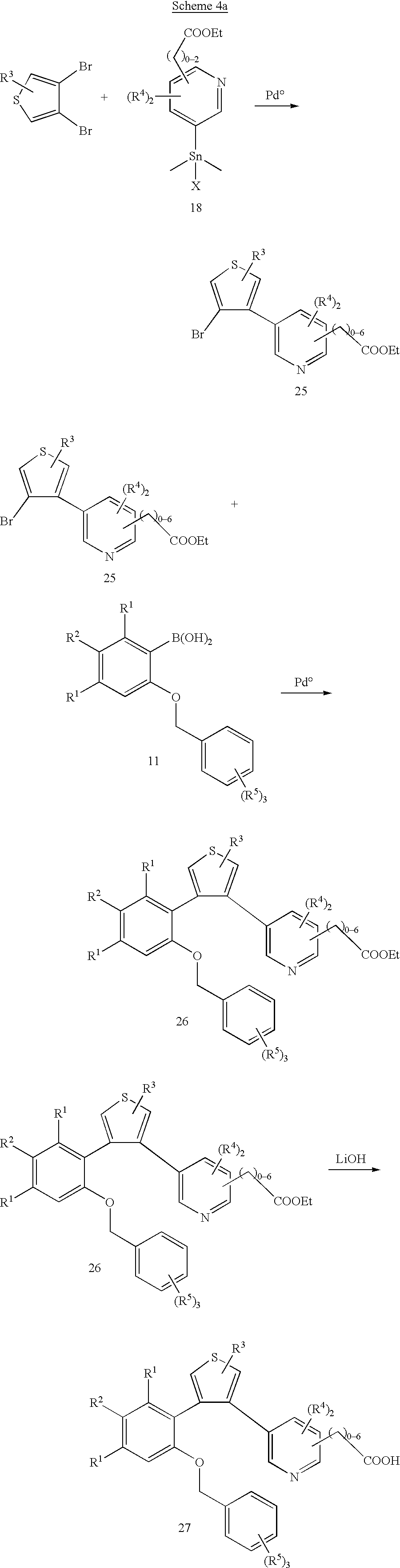

Assays for Determining Biological Activity

The compounds of formula I can be tested using the following assays to demonstrate their prostanoid antagonist or agonist activity in vitro and in vivo and their selectivity. The prostaglandin receptors investigated are DP, EP₁, EP₂, EP₃, EP₄, FP, IP and TP.

Stable Expression of Prostanoid Receptors in the Human Embryonic Kidney (HEK) 293(ebna) Cell Line

Prostanoid receptor cDNAs corresponding to full length coding sequences were subcloned into the appropriate sites of mammalian expression vectors and transfected into HEK 293(ebna) cells. HEK 293(ebna) cells expressing the individual cDNAs were grown under selection and individual colonies were isolated after 2-3 weeks of growth using the cloning ring method and subsequently expanded into clonal cell lines.

Prostanoid Receptor Binding Assays

HEK 293(ebna) cells are maintained in culture, harvested and membranes are prepared by differential centrifugation, following lysis of the cells in the presence of protease inhibitors, for use in receptor binding assays. Prostanoid receptor binding assays are performed in 10 mM MES/KOH (pH 6.0) (EPs, FP and TP) or 10 mM HEPES/KOH (pH 7.4) (DP and IP), containing 1 mM EDTA, 10 mM divalent cation and the appropriate radioligand. The reaction is initiated by addition of membrane protein. Ligands are added in dimethylsulfoxide which is kept constant at 1% (v/v) in all incubations. Non-specific binding is determined in the presence of 1 μM of the corresponding non-radioactive prostanoid. Incubations are conducted for 60 min at room temperature or 30° C. and terminated by rapid filtration. Specific binding is calculated by subtracting non specific binding from total binding. The residual specific binding at each ligand concentration is calculated and expressed as a function of ligand concentration in order to construct sigmoidal concentration-response curves for determination of ligand affinity.

Prostanoid Receptor Agonist and Antagonist Assays

Whole cell second messenger assays measuring stimulation (EP₂, EP₄, DP and IP in HEK 293(ebna) cells) or inhibition (EP₃ in human erythroleukemia (HEL) cells) of intracellular cAMP accumulation or mobilization of intracellular calcium (EP₁, FP and TP in HEK 293(ebna) cells. For cAMP assays, cells are harvested and resuspended in HBSS containing 25 mM HEPES, pH 7.4. Incubations contain 100 μM RO-20174 (phosphodiesterase type IV inhibitor, available from Biomol) and, in the case of the EP₃ inhibition assay only, 15 μM forskolin to stimulate cAMP production. Samples are incubated al 37° C. for 10 min, the reaction is terminated and cAMP levels are then measured. For calcium mobilization assays, cells are charged with the co-factors reduced glutathione and coelenterazine, harvested and resuspended in Ham's F12 medium. Calcium mobilization is measured by monitoring luminescence provoked by calcium binding to the intracellular photoprotein aequorin. Ligands are added in dimethylsulfoxide which is kept constant at 1% (v/v) in all incubations. For agonists, second messenger responses are expressed as a function of ligand concentration and both EC₅₀ values and the maximum response as compared to a prostanoid standard are calculated. For antagonists, the ability of a ligand to inhibit an agonist response is determined by Schild analysis and both K_{B} and slope values are calculated.

In Vivo Micturition Assay

Anesthesia, Cannulation and Continuous Infusion Cystometry.

Female Wistar rats (200-250 g) were anesthetized with urethane (1.2 g/kg i.p.). The femoral artery and vein were cannulated with polyethylene tubes through a small cut-down. A midline incision in the abdomen was used to expose the bladder. The bladder was cannulated through the dome using a commercial i.v. cannulation system (e.g. Surflow) in which a plastic or Teflon cannula is carried on a needle used as a trocar. A purse-string suturing was required so that the seal is watertight. Continuous bladder infusion with sterile 0.9% saline (0.05-0.1 ml/min, Harvard infusion pump) demonstrated the normal micturition pattern consisting of low filling pressure and flat filling phase, and abrupt rise pressure rise during the micturition episode with fluid flow from the meatus.

Data Collection/Analysis

Bladder pressure will be monitored using a Statham-type transducer coupled through a preamplifier to a MacLab A/D interface unit. Zero pressure is taken to be the tabletop (anesthetized) or observation cage floor (conscious). Cystometrogram characteristics (number of micturitions, micturition interval, volume threshold and micturition pressure) were meaured over 30 minute periods and compared for the pre-drug and drug treatment conditions.

Results

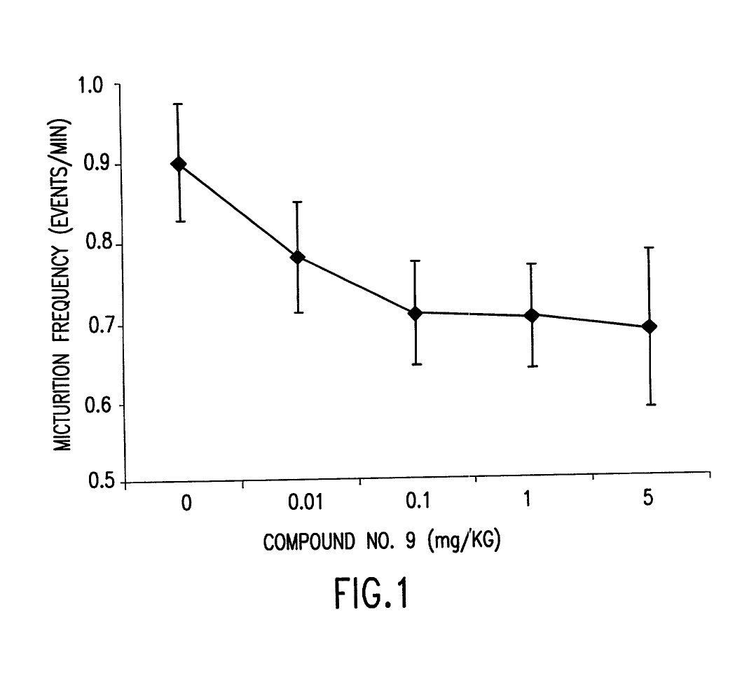

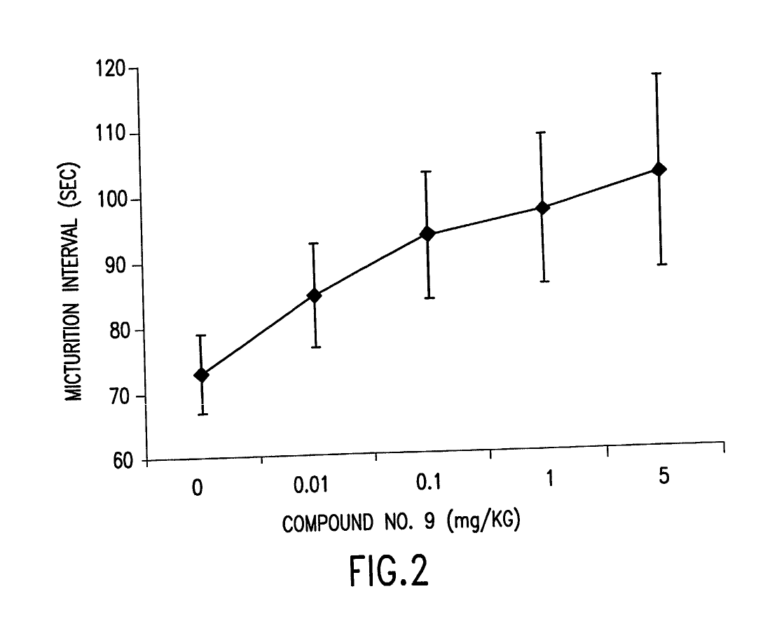

As shown in FIGS. 1 and 2, the EP1 antagonist dose-dependently reduced the number of micturition events/min and increased the micturition interval.

### EXAMPLES

The invention is illustrated in connection with the following non-limiting Examples. All the end products of the formula I were analyzed by NMR, TLC and mass spectrometry.

Intermediates were analyzed by NMR and TLC.

Most compounds were purified by flash chromatography on silica gel.

Recrystallization and/or swish (suspension in a solvent followed by filtration of the solid) with a solvent such as ether:hexane 1:1.

The course of reactions was followed by thin layer chromatography (TLC) and reaction times are given for illustration only.

Temperatures are in degrees Celsius.

### Example 1

### 4-{3-[2-(Phenylmethoxy)Phenyl]-2-Thienyl}Benzoic Acid

2-Bromophenylbenzyl ether (3.5 g, 13.3 mmol, prepared from 2-bromophenol following standard benzylation procedure) was reacted with thiophene 3-boronic acid (2.1 g, 16.6 mmol, purchased from Lancaster), tetrakis(triphenylphosphine) palladium (770 mg, 0.7 mmol) and 2M Na₂CO₃ (25 mL) in 1,2-dimethoxyethane (75 mL) at 90° C for 24 hours. The mixture was cooled down and a saturated solution of ammonium chloride and ethyl acetate were added. The separated aqueous layer was extracted with ethyl acetate (3×50 mL) and the combined organic layers were dried (MgSO₄ anh.), filtered and evaporated. Flash-chromatography of the residue (ethyl acetate-hexanes 1:10) yielded 3.5 g of thiophene 3 (Scheme 1) which was selectively brominated at the 2 position according to the following procedure: Thiophene 3 (2.4 g, 9.0 mmol) was treated with N-bromosuccinimide (1.6 g, 9.0 mmol) in THF (50 mL) containing 0.5 mL of water. The mixture was stirred at room temperature for 1.5 hours and water and diethyl ether were added. The separated aqueous layer was extracted with ether (3×50 mL) and the combined organic layers were dried (MgSO₄ anh.), filtered and evaporated. Flash-chromatography of the residue (ethyl acetate-hexanes 1:10) yielded 3.1 g of the bromothiophene 4. The 2-bromo thiophene derivative 4 (3.1 g, 9.0 mmol) was reacted with 4-carboxybenzeneboronic acid (1.5 g, 9.1 mmol), tetrakis(triphenylphosphine) palladium (520 mg, 0.5 mmol) and 2M Na₂CO₃ (14 mL) in 1,2-dimethoxyethane (100 mL) at 90° C. for 24 hours. The mixture was cooled down and a saturated solution of ammonium chloride and ethyl acetate were added. The separated aqueous layer was extracted with ethyl acetate (3×50 mL) and the combined organic layers were dried (MgSO₄ anh.), filtered and evaporated. Flash-chromatography of the residue (ethyl acetate-hexanes 1:10) yielded 1.9 g of the title compound. ¹H nmr (400 MHz, CD₃COCD3) δ ppm 7.87 (2H, d, J=11.5 Hz), 7.58 (1H, d, J=5.0 Hz), 7.35-7.09 (11H, m), 6.96 (1H, dt, J=10.0, 1.5 Hz), 4.95 (2H, s). Elemental analysis calculated for C₂₄H₁₇SO₃Na. 1.5H₂O: C, 66.20, H, 4.63, S, 7.36, found: C, 66.00, H, 4.27, S, 7.44.

### Example 2

4-{2-[2-(Phenylmethoxy)Phenyl]-3-Thienyl}Benzoic Acid

A mixture of 3-bromothiophene (725 mg, 7.7 mmol) (Scheme 2), carboethoxybenzeneboronic acid (1.1 g, 5.9 mmol), tetrakis(triphenylphosphine) palladium (444 mg, 0.4 mmol) and 2M Na₂CO₃ (9 mL) in 1,2-dimethoxyethane (60 mL) was heated at 90° C for 24 hours. The mixture was cooled down and a saturated solution of ammonium chloride and ethyl acetate were added. The separated aqueous layer was extracted with ethyl acetate (3×50 mL) and the combined organic layers were dried (MgSO₄ anh.), filtered and evaporated. Flash-chromatography of the residue (ethyl acetate-hexanes 1:5) yielded 800 mg of thiophene derivative 9 which was converted to the bromide 10 using the conditions described above. The bromide 10 (1.3 g, 4.0 mmol) was treated with the boronic acid 11 (1.3 g, 6.0 mmol), tetrakis(triphenylphosphine) palladium (230 mg, 0.2 mmol) and 2M Na₂CO₃ (1.2 mL) in 1,2-dimethoxyethane (25 mL) at 90° C. for 24 hours. The mixture was cooled down and a saturated solution of ammonium chloride and ethyl acetate were added. The separated aqueous layer was extracted with ethyl acetate (3×50 mL) and the combined organic layers were dried (MgSO₄ anh.), filtered and evaporated. Flash-chromatography of the residue (ethyl acetate-hexanes 1:10) yielded 700 mg of the ester which was then heated at 50° C for 5 hours in a (1:1) mixture of dioxane-water (10 mL total) in the presence of lithium hydroxide (210 mg). Work-up afforded 456 mg of the title compound. ¹H nmr (400 MHz, CD₃COCD₃) δ ppm 8.11 (2H, d, J=11.5 Hz), 7.89 (2H, d, J=11.5 Hz), 7.63 (2H, d, J=11.5 Hz), 7.66 (1H, d, J=7.5 Hz), 7.59 (1H, d, J=7.5 Hz), 7.32, 7.22 (4H, 2 m), 7.08 (2H, m), 6.97 (1H, dt, J=10.0, 1.5 Hz), 4.95 (2H, s). Elemental analysis calculated for C₂₄H₁₇SO₃Na.1.5H₂O: C, 66.20, H, 4.63, S, 7.36; found:

### Example 3

### 2-(4-{3-[2-(Phenylmethoxy)Phenyl]-2-Thienyl}Phenyl)Acetic Acid

Prepared following the standard procedure described in example 1. ¹H nmr (400 MHz, CD₃COCD₃) δ ppm 7.47 (1H, d, J=7.5 Hz), 7.30-7.06 (14H, m), 6.91 (1H, t, J=7.5 Hz), 4.95 (2H, s), 3.60 (2H, s).

### Example 4

### (4-{3-[2-(Phenylmethoxy)Phenyl]-2-Thienyl}Phenyl)Methan-1-ol

To a solution of ethyl ester of example 1 (39 mg, 0.096 mmol) in THF (1.5 mL) at −78° C. was added Et₃BHLi (1M in THF, 0.38 mL, 0.38 mmol) and the reaction was warmed to room temperature and stirred for 1 h. The mixture was quenched with saturated NH₄Cl, diluted with Et₂O and washed successively with HCl 10%, aq. NaHCO₃ and brine. The ether layer was dried with anh. MgSO₄, filtered and concentrated under reduced pressure to give a residue which was purified by flash chromatography (75% Hexanes/25% AcOEt) to provide the title compound (28.3 mg). ¹H nmr (400 MHz, acetone-d₆) δ ppm 7.45 (1H, d, J=7.5 Hz), 7.32-7.05 (13H, m), 6.91 (1H, t, J=7.5 Hz), 4.97 (2H, s), 4.60 (2H, d, J=7.5 Hz), 3.95 (1H, t, J=7.5 Hz).

### Example 5

### 2-(4-{3-[2-(Phenylmethoxy)Phenyl]-2-Thienyl}Phenyl) Propan-2-ol

To a solution of ethyl ester of example 1 (34 mg, 0.084 mmol) in ether (1.5 mL) at −78° C. was added MeMgBr (3M in ether, 0.11 mL, 0.33 mmol) and the reaction was warmed to room temperature and stirred for 1 h. The mixture was quenched with saturated NH₄Cl, diluted with Et₂O and washed successively with HCl 10%, aq. NaHCO₃ and brine. The ether layer was dried with anh. MgSO₄, filtered and concentrated under reduced pressure to give a residue which was purified by flash chromatography (75% Hexanes/25% AcOEt) to provide the title compound (25.6 mg). ¹H nmr (400 MHz, acetone-d₆) δ ppm 7.45-7.35 (3H, m), 7.39-7.05 (11H, m), 6.91 (1H, t, J=7.5 Hz), 4.95 (2H, s), 3.95 (1H, bs), 1.47 (6H, s).

### Example 6

### 1-(4-{3-[2-(Phenylmethoxy)Phenyl]-2-Thienyl}Phenyl)Ethan-1-ol

To a solution of the alcohol of example 4 (200 mg, 0.536 mmol) in CH₂Cl₂ is added MnO₂ (467 mg, 5.36 mmol) and the mixture is stirred overnight. The reaction is then filtred throught a plug of celite and the crude mixture diluted in ether (5.0 mL) and cooled at −78° C. MeMgBr was then added (3M in ether, 0.22 mL, 0.66 mmol) and the reaction was warmed to room temperature and stirred for 1 h. The mixture was quenched with saturated NH₄Cl, diluted with Et₂O and washed successively with HCl 10%, aq. NaHCO₃ and brine. The ether layer was dried with anh. MgSO₄, filtered and concentrated under reduced pressure to give a residue which was separated in two and one part was purified by flash chromatography (70% Hexanes/30% AcOEt) to provide the title compound (19.4 mg). ¹H nmr (400 MHz, acetone-d₆) δ ppm 7.45 (1H, d, J=7.5 Hz), 7.32-7.05 (13H, m), 6.91 (1H, t, J=7.5 Hz), 4.97 (2H, s), 4.80 (1H, m) 4.12 (1H, d, J=5.0 Hz), 3.95 (3H, d, J=7.5 Hz).

### Example 7

### 4-{5-Bromo-3-[2-(Phenylmethoxy)Phenylmethoxy)Phenyl]-2-Thenyl} Benzoic Acid

To a solution of example 1 (0.48 g, 1.2 mmol) in THF/water (10/0.1 mL) was added N-bromosuccinimide (0.22 g, 1.2 mmol). The reaction was stirred overnight at room temperature. The mixture was then diluted with AcOEt and water, and the combined organic layer was washed with HCl 10%, NaHCO₃ (aq), brine, drier (anh. MgSO₄) and concentrated under reduced pressure to give a residue which was purified by flash chromatography (95% CH₂Cl₂/5% AcOEt) and crystalisation (CH₂Cl₂/Hexanes) to provide the title compound (0.16 g). ¹H nmr (400 MHz, acetone-d₆) δ ppm 7.76 (2H, d, J=8.38 Hz), 7.23-7.11 (7H, m), 6.95 (2H, m), 6.87 (2H, d, J=8.16 Hz), 6.71 (1H, t, J=7.5 Hz), 4.90 (2H, s). Elemental analysis calculated for C₂₄H₁₆BrNaSO₃.4 H₂O: C, 51.53; H, 4.32, S, 5.73: found: C, 51.14; H, 3.76, S, 5.52.

### Example 8

### 4-{3-[5-Chloro-2-(Phenylmethoxy)Phenyl]-2-Thienyl} Benzoic Acid

Prepared following the procedure described in example 1. ¹H nmr (400 MHz, CD₃COCD₃) δ ppm 7.90 (2H, d, J=11.5 Hz), 7.58 (1H, d, J=5.0 Hz), 7.38-7.05 (1H, m), 4.94 (2H, s). Elemental analysis calculated for C₂₄H₁₆ClSO₃Na.H₂O: C, 62.54; H, 3.94, S, 6.96; found: C, 62.14; H, 3.82; S, 6.38.

### Example 9

### 3-{3-[5-Chloro-2-(Phenylmethoxy)Phenyl]-2-Thienyl} Benzoic Acid

Prepared following the procedure described in example 1. ¹H nmr (400 MHz, CD₃COCD₃) δ ppm 7.56-7.10 (14H, m), 5.13 (2H, s).

### Example 10

### 2-Chloro-5{-3-[5-Chloro-2-(Phenylmethoxy)Phenyl](2-Thienyl)} Benzoic Acid

Prepared following the procedure described in example 1. ¹H nmr (400 MHz, acetone-d₆) δ ppm 7.80 (1H, d, J=2.2 Hz), 7.57 (1H, d, J=5.22 Hz), 7.40-7.20 (8H, m), 7.12-7.07 (3H, m), 4.92 (2H, s). Elemental analysis calculated for C₂₄H₁₅Cl₂NaSO₃.4 H₂O: C, 52.46; H, 4.22, S, 5.84; found: C, 52.05; H, 3.95, S, 5.54.

### Example 11

### 2-[2-(3-(2H-1,2,3,4-Tetraazol-5-yl)Phenyl)(3-Thienyl)]-4-Chloro-1-(Phenylmethoxy)Benzene

To a solution of nitrile corresponding to the acid of example 9 (0.10 g, 0.26 mmol) in N-methyl pyrrolidine was added pyridine hydrochloride (0.30 g, 2.6 mmol) and sodium azide (0.34 g, 5.2 mmol). The reaction was stirred for 36 h. at 135° C., cooled down and then quenched by adding HCl 10%, diluted with AcOEt, washed with HCl 10% , brine, dried (anh. MgSO₄) and concentrated under reduced pressure to give a residue which was purified by flash-chromatography (80% CH₂Cl₂/19% AcOEt/1% AcOH), then crystalised (CH₂Cl₂/Hexanes to provide the title compound (0.05 g). ¹H nmr (400 MHz, acetone-d₆) δ ppm 8.10 (1H, s), 8.01 (1H, d, J=7.5 Hz), 7.57 (1H, d, J=5.22 Hz), 7.45 (1H, t,J=7.5 Hz), 7.33 (1H, d, J=7.5 Hz), 7.30 (1H, d, J=7.5 Hz), 7.25-7.12 (5H, m), 7.07 (3H, m), 4.92 (2H, s).

### Example 12

### 5-{3-[5-Chloro-2-(Phenylmethoxy)Phenyl](2-Thienyl)}-2-Methoxybenzoic Acid

Prepared following the procedure described in example 1. ¹H nmr (400 MHz, acetone-d₆) δ ppm 7.87 (1H, d, J=2.30 Hz), 7.48 (1H, d, J=5.22 Hz), 7.37 (1H, dd, J=8.80, 2.70 Hz), 7.32-7.20 (4H, m), 7.18-7.05 (6H, m), 4.97 (2H, s), 3.95 (3H, s). Elemental analysis calculated for C₂₅H₁₈ClNaSO₄.3H₂O: C, 56.98; H, 4.59, S, 6.08: found: C, 56.86; H, 3.95, S, 5.50.

### Example 13

### 3-{3-[5-Chloro-2-(Phenylmethoxy)Phenyl](2-Thienyl)}-4-Fluorobenzoic Acid

Prepared following the procedure described in example 1. ¹H nmr (400 MHz, acetone-d₆) δ ppm 8.00 (2H, m), 7.65 (1H, d, J=5.20 Hz), 7.30-7.10 (9H, m), 7.03 (1H, d, J=8.0 Hz), 4.95 (2H, s), 1.97 (3H, s). Elemental analysis calculated for C₂₄H₁₅ClFNaSO₃.2 H₂O: C, 58.01; H, 3.85: found: C, 58.01; H, 3.40.

### Example 14

### 3-{3-[5-Chloro-2-(Phenylmethoxy)Phenyl]-2-Thienyl}Benzamide

To a solution of the acid of example 9 1(0.45 g, 1.1 mmol) in pyridine (10 mL) at 0° C. was added methanesulfonyl chloride (0.10 mL, 1.3 mmol) and the reaction was stirred for 1 h. at 0° C. Ammonia was then introduced and the mixture was stirred for 1 h. at room temperature. The mixture was slowly quenched by adding HCl 10%, diluted with AcOEt, washed with HCl 10% , NaHCO₃ (aq), brine, dried (anh. MgSO₄) and concentrated under reduced pressure to give a residue which was purified by crystalisation (AcOEt/Hexanes) to provide the title compound (0.4 g). ¹H nmr (400 MHz, acetone-d₆) δ ppm 7.92 (1H, s), 7.81 (1H, d, J=3.57 Hz), 7.54 (1H, d, J=5.17 Hz), 7.38 (1H, bs), 7.31-7.23 (6H, m), 7.18 (1H, d, J=5.10 Hz) 7.15-7.02 (4H, m), 6.62 (1H,bs), 4.95 (2H, s). Elemental analysis calculated for C₂₄H₁₈ClNSO₂.H₂O: C, 65.82; H, 4.60; N, 3.20; S, 7.32; found: C, 66.21; H, 4.23; N, 3.00; S, 7.53.

### Example 15

### 2-(3-{3-[5-Chloro-2-(Phenylmethoxy)Phenyl]-2-Thienyl}Phenyl)Acetic Acid

Prepared following the procedure described in example 1. ¹H nmr (400 MHz, acetone-d₆) δ ppm 7.49 (1H, d, J=5.2 Hz), 7.29-7.05 (13H, m), 4.91 (2H, s), 3.52 (2H, s). Elemental analysis calculated for C₂₅H₁₈ClNaSO₃.1.5 H₂O: C, 62.04; H, 4.37, S, 6.62: found: C, 61.63; H, 4.14, S, 6.37.

### Example 16

### 4-{3-[5-Chloro-2-(Phenylmethoxy)Phenyl](2-Thienyl)}-2-Methylbenzoic Acid

Prepared following the procedure described in example 1. ¹H nmr (400 MHz, CD₃COCD₃) δ ppm 7.80 (1H, d, J=10.5 Hz), 7.50 (1H, d, J=7.0 Hz), 7.28-7.00 (11H, m), 4.88 (2H, s), 2.05 (3H, s). Elemental analysis calculated for C₂₅H₁₈ClSO₃Na.1.5H₂O: C, 62.05, H, 4.37, S, 6.63; found: C, 62.34, H, 4.00, S, 6.16.

### Example 17

4-(3-{2-[(2-Chloro-4-Fluorophenyl)Methoxy]-5-Nitrophenyl}-2-Thienyl)Benzoic Acid

Prepared following the procedure described in example 1. ¹H nmr (400 MHz, acetone-d₆) δ ppm 8.28 (1H, dd, J=9.13, 2.88 Hz), 8.10 (1H, d, J=2.87 Hz), 7.87 (2H, d, J=8.52 Hz), 7.64 (1H, d, J=5.20 Hz), 7.39-7.14 (6H, m), 7.05 (1H, dt, J=8.44, 2.60 Hz), 5.14 (2H, s). Elemental analysis calculated for C₂₄H₁₄FClNNaSO₅.2 H₂O: C,53.19; H, 3.34; N, 2.58; S, 5.92: found: C,52.71; H, 2.90; N, 2.52; S, 5.67.

### Example 18

### (4-{3-[5-Chloro-2-(Phenylmethoxy)Phenyl](2-Thienyl)}Phenyl)-N-(3-Pyridylmethyl)Formamide

The benzoic acid of example 8 (900 mg, 2.2 mmol) was added at −78° C. to a mixture of EDCI (820 mg, 4.4 mmol) and 3-aminomethylpyridine (330 μL, 3.0 mmol) in dichloromethane (20 mL). The mixture was warmed to room temperature and stirred for 12 hours, and a 10% solution of sodium bicarbonate was added. The separated aqueous layer was extracted with dichloromethane (3×50 mL) and the combined organic layers were dried (MgSO₄ anh.), filtered and evaporated. Flash-chromatography of the residue (ethyl acetate: dichloromethane, 3:7) yielded 450 mg of the title compound. ¹H nmr (400 MHz, CD₃COCD₃) δ ppm 8.60 (1H, br. s.), 8.46 (1H, m), 8.31 (1H, m), 7.82 (2H, d, J=11.5 Hz), 7.77 (1H, d, J=11.5 Hz), 7.56 (1H, d, J=7.5 Hz), 7.31-7.16 (9H, m), 7.06 (3H, m), 4.94 (2H, s), 4.62 (2H, br. d). Elemental analysis calculated for C₃₀H₂₄ClN₂SO₂Na.H₂O: C, H, S; found: C, H, S.

### Example 19

### [4-(3-{2-[(2-Chloro-4-Fluorophenyl)Methoxy]-5-Nitrophenyl}(2-Thienyl))PHENYL]-N-(3-Pyridylmethyl)Formamide

Prepared following the procedure described for example 18. ¹H nmr (400 MHz, acetone-d₆) δ ppm 8.78 (1H, s), 8.68 (1H, bs), 8.63 (1H, d, J=3.8 Hz), 8.35 (1H, d, J=8.16 Hz), 8.27 (1H, dd, J=9.13, 2.90 Hz), 8.08 (1H, d, J=2.86 Hz), 7.79 (3H, m), 7.62 (1H, d, J=5.20 Hz), 7.37 (1H, d, J=9.13 Hz), 7.27 (5H, m), 7.08 (1H, m), 5.13 (2H, s), 4.75 (2H, d, J=5.71 Hz).

### Example 20

### (4-{3-[5-Chloro-2-(Phenylmethoxy)Phenyl](2-Thienyl)}Phenyl)-N-(2-Thienylsulfonyl)Formamide

The benzoic acid of example 8 (350 mg, 0.8 mmol) was added at room temperature to a mixture of EDCI (175 mg, 0.9 mmol)l, 4-dimethylaminopyridine (300 mg, 2.5 mmol) and 2-sulfonamidothiophene (156 mg, 1.0 mmol) in dichloromethane (12 mL). The mixture was stirred for 12 hours at room temperature and a IN solution of HCl was added. The separated aqueous layer was extracted with dichloromethane (3×50 mL) and the combined organic layers were dried (MgSO₄ anh.), filtered and evaporated. Flash-chromatography of the residue (ethyl acetate: dichloromethane, 3:7) was followed by trituration in dichloromethane:hexanes (1:10) yielded 290 mg of the title compound. ¹H nmr (400 MHz, CD₃COCD₃) δ ppm 7.99 (1H, dd, J=5.5, 1.5 Hz), 7.91 (1H, m), 7.81 (2H, d, J=11.5 Hz), 7.60 (1H, d, J=7.5 Hz), 7.31 (2H, d, J=10.0 Hz), 7.29 (1H, m), 7.20 (6H, m), 7.10 (1H, d, J=11.5 Hz), 7.06 (2H, m), 4.91 (2H, s). Elemental analysis calculated for C₂₈H₁₉ClNS₃O₄Na.1.5H₂O: C, 54.67, H, 3.61, S, 15.64, N, 2.28; found: C, 54.28, H, 3.24, S, 15.60, N, 2.29.

### Example 21

### 4-(3-{2-[(2,4-Difluorophenyl)Methoxy]-5-Chlorophenyl}(2-Thienyl))-3-Methylbenzo]C Acid

Prepared following the procedure described in example 1. ¹H nmr (400 MHz, acetone-d₆) δ ppm 7.77 (2H, m), 7.50 (2H,m), 7.29-7.17 (4H, m), 7.07 (2H, m), 6.93 (1H, d, J=2.3 Hz), 5.09 (2H, s), 1.97 (3H, s). Elemental analysis calculated for C₂₅H₁₆ClF₂NaSO₃.2.5 H₂O: C, 55.82; H,3.93; S, 5.96: found: C, 55.61; H, 3.41; S, 5.87.

### Example 22

### 4-(3-{5-Chloro-2-[(4-Fluorophenyl)Methoxy]Phenyl}(2-Thienyl))-3-Methylbenzoic Acid

Prepared following the procedure described in example 1. ¹H nmr (400 MHz, acetone-d₆) δ ppm 7.80 (2H, m), 7.60 (1H, d, J=5.2 Hz), 7.35-7.25 (4H, m), 7.20 (1H, dd, J=7.5, 2.2Hz), 7.12-7.02 (3H, m), 6.98 (1H, d, J=2.2 Hz), 5.03 (2H, s), 2.05 (3H, s). Elemental analysis calculated for C₂₅H₁₇ClFNaSO₃.1.5 H₂O: C, 59.82; H, 4.01: found: C, 59.74; H, 3.67.

### Example 23

### 4-{3-[5-Chloro-2-(Phenylmethoxy)Phenyl](2-Thienyl)}-3-Methylbenzoic Acid

Prepared following the procedure described in example 1. ¹H nmr (400 MHz, acetone-d₆) δ ppm 7.80 (2H, m), 7.58 (1H, d, J=5.2 Hz), 7.35-7.15 (8H, m), 7.04-6.98 (2H, m), 5.03 (2H, s), 2.05 (3H, s). Elemental analysis calculated for C₂₅H₁₈ClNaSO₃.1 H₂O: C, 63.22; H, 4.24; S, 6.75: found: C, 63.11; H, 4.04; S, 6.62.

### Example 24

### 4-{4-[2-(Phenylmethoxy)Phenyl]-3-Thienyl}Benzoic Acid

3,4-bromothiophene (920 μL, 7.7 mmol) was treated with 8 (500 mg, 2.6 mmol), tetrakis(triphenylphosphine) palladium (150 mg, 0.15 mmol) and 2M Na₂CO₃ in 1,2-dimethoxyethane (25 mL) at 90° C. for 12 hours. The mixture was cooled and a saturated solution of ammonium chloride and ethyl acetate were added. The separated aqueous layer was extracted with ethyl acetate (3×50 mL) and the combined organic layers were dried (MgSO₄ anh.), filtered and evaporated. Flash-chromatography of the residue (ethyl acetate: hexanes, 1:8) yielded 420 mg of 15 which was then treated with boronic acid 11 following a procedure similar to those already described (vide supra) to provide 16. Ethyl ester 16 (560 mg, 1.4 mmol) was then hydrolyzed with IN lithium hydroxide (8 mL) in dioxane (16 mL) at 50° C for 5 hours. The mixture was cooled down and a IN solution of HCl and ethyl acetate were added. The separated aqueous layer was extracted with ethyl acetate (3×50 mL) and the combined organic layers were dried (MgSO₄ anh.), filtered and evaporated. Flash-chromatography of the residue (dichloromethane: ethyl acetate: acetic acid, 95:4:1) provided the title compound. ¹H nmr (400 MHz, acetone-d₆) δ ppm 7.84 (2H, d, J=8.1 Hz), 7.63 (1H, d, J=3.2 Hz), 7.46 (1H, d,J=3.1 Hz), 7.29-7.20 (7H, m), 6.99-6.93 (4H, m), 4.76 (2H, s). Elemental analysis calculated for C₃₀H₂₅N₂SClO₂.1 H₂O: C, 67.59; H, 4.49: found: C, 67.54; H, 4.33.

### Example 25

### (4-{4-[2-(Phenylmethoxy)Phenyl](3-Thienyl)}Phenyl)-N-(3-Pyridylmethyl)Formamide

Prepared following the standard procedure described for example 18 1H nmr (400 MHz, CD₃COCD₃) δ ppm 8.60 (1H, d, J=1.7 Hz), 8.45 (1H, dd, J=4.8, 1.6 Hz), 8.30 (1H, bs), 7.75 (3H, m), 7.60 (1H, d, J=3.3 Hz), 7.45 (1H, d, J=3.3 Hz), 7.23 (8H, m), 6.95 (4H, m), 4.77 (2H, s), 4.60 (211, d, J=6.1 Hz ). Elemental analysis calculated for C₃₀H₂₅N₂SClO₂.0.5 H₂O: C, 69.02; H, 5.02; N, 5.37; S, 6.15: found: C, 68.51; H, 5.06; N, 5.39; S, 6.62.

### Example 26

### 4-[3-(2-}[4-(Difluoromethoxy)Phenyl]Methoxy}-5-Chlorophenyl)(2-Thienyl)]-3-Methylbenzoic Acid

Prepared following the standard procedure described in example 1 1H nmr (400 MHz, CD₃COCD₃) δ ppm 7.82 (1H, s), 7.80 (1H, d, J=11.5 Hz), 7.60 (1H, d, J=7.5 Hz), 7.32 (4H, m), 7.20 (1H, dd, J=11.5, 1.5 Hz), 7.18 (2H, m), 7.09 (1H, d, J=11.5 Hz), 6.97 (1H, s), 5.06 (2H, s).

### Example 27

### 4-(3-{2-[(4-Carboxyphenyl)Methoxy]-5-Chlorophenyl}-2-Thienyl)Benzoic Acid

Prepared following the standard procedure described in example 1 ¹H nmr (400 MH, CD₃COCD₃) δ ppm 7.88 (4H, t. J=7.0 Hz), 7.62 (1H, d, J=6.0 Hz), 7.31 (3H, m), 7.19 (4H, m), 7.10 (1H, d, J=11.5 Hz), 5.04 (2H, s). Elemental analysis calculated for C₂₅H₁₅ClN₂SO₅Na.6H₂O: C, 48.67, H, 4.41; found: C, 49.00, H, 3.50.

### Example 28

### 3-(3-{2-[(4-Carboxyphenyl)Methoxy]-5-Chlorophenyl}-2-Thienyl)Benzoic Acid

Prepared following the standard procedure described in example 1 ¹H nmr (400 MHz, CD₃COCD₃) δ ppm 7.98 (1H, s.), 7.92 (3H, m), 7.59 (1H, d, J=7.5 Hz), 7.45 (1H, m), 7.40 (1H, t, J=10.0 Hz), 7.33 (1H, dd,J=11.5.1.5 Hz), 7.21 (4H, m), 7.09 (1H, d, J=11.5 Hz), 5.01 (2H, s).

### Example 29

### 4-(3-{5-Chloro-2-[(2-Chloro-4-Fluorophenyl)Methoxy]Phenyl}(2Thienyl))-3-Methylbenzoic Acid

Prepared following the standard procedure described in example 1 ¹H nmr (400 MHz, CD₃COCD₃) δ ppm 7.79 (2H, m), 7.59 (1H, d, J=5.2 Hz), 7.36-7.05 (7H, m), 7.00 (1H, d, J=2.68 Hz), 5.07 (2H, s), 2.04 (3H, s). Elemental analysis calculated for C₂₅H₁₆Cl₂FNaSO₃.H₂O: C, 56.94; H, 3.44; S, 6.08: found: C, 57.18; H, 3.40; S, 6.33.

### Example 30

### [4-(3-{5-Chloro-2-[(2-Chloro-4-Fluorophenyl)Methoxy]Phenyl}(2-Thienyl))-3-Methylphenyl]-N-(3-Pyridylmethyl)Formamide

Prepared following the standard procedure described for example 18 ¹H nmr (400 MHz, acetone-d₆) δ ppm 8.61 (1H, s), 8.42 (2H, m), 7.80-7.65 (3H, m), 7.53 (1H, d, J=5.18 Hz), 7.45-7.00 (9H, m), 5.05 (2H, s), 4.60 (2H, d, J=7.5 Hz), 2.00 (3H, s). Elemental analysis calculated for C₃₁H₂₄N₂Cl₃SFO₂.0.5H₂O: C, 59.77; H, 4.05; N, 4.50; S, 5.15: found: C, 59.91; H, 4.22; N, 4.47; S, 5.38.

### Example 31

### 5-{3-[5-Chloro-2-(Phenylmethoxy)Phenyl]-2-Thienyl}Pyridine-3-Carboxylic Acid (1a)

2-Bromophenylbenzyl ether (3.5 g, 13.3 mmol, prepared from 2-bromophenol following standard benzylation procedure ) was reacted with thiophene 3-boronic acid (2.1 g, 16.6 mmol, purchased from Lancaster), tetrakis(triphenylphosphine) palladium (770 mg, 0.7 mmol) and 2M Na₂CO₃ (25 mL) in 1,2-dimethoxyethane (75 mL) at 90° C for 24 hours. The mixture was cooled down and a saturated solution of ammonium chloride and ethyl acetate were added. The separated aqueous layer was extracted with ethyl acetate (3×50 mL) and the combined organic layers were dried (MgSO₄ anh.), filtered and evaporated. Flash-chromatography of the residue (ethyl acetate-hexanes 1:10) yielded 3.5 g of thiophene 3 (Scheme 1) which was selectively brominated at the 2 position according to the following procedure:

Thiophene 3 (2.4 g, 9.0 mmol) was treated with N-bromosuccinimide (1.6 g, 9.0 mmol) in THF (50 mL) containing 0.5 mL of water. The mixture was stirred at room temperature for 1.5 hours and water and diethyl ether were added. The separated aqueous layer was extracted with ether (3×50 mL) and the combined organic layers were dried (MgSO₄ anh.), filtered and evaporated. Flash-chromatography of the residue (ethyl acetate-hexanes 1:10) yielded 3.1 g of the bromothiophene 19.

To a solution of hexamethylditin (14.22 g, 43.4 mmol) and ethyl 5-bromonicotinate (10.0 g, 43.4 mmol), in toluene (400 ml) is added Pd(PPh₃)4 (2.54 g, 2.2 mmol). The reaction is stirred 3 h at 100° C. then cooled down and evaporated under reduced pressure to give a residu which is purified by flash chromatography (90% Hexanes/10% AcOEt) to afford the desired material 18 (11.06 g, 81%).

To a solution of stannane 18 (10.0 g, 31.85 mmol) and bromo derivative 19 (10.52 g, 27.69 mmol), in DMF (150 ml) is added Pd₂(dba)₃ (1.27 g, 1.38 mmol), and AsPh₃ (3.39 g, 11.08 mmol). The reaction is stirred at 100° C. overnight, cooled down, diluted with AcOEt, washed with water, brine, MgSO₄ and concentrated under reduced pressure to give a residu which is purified by flash chromatography (60% CH₂Cl₂/40% Hexanes to 80% CH₂Cl₂/20% AcOEt) to provide ethyl ester 20 (8.18 g, 66%) which is hydrolyzed under basic conditions to provide the title compound. ¹H nmr (400 MHz, CD₃COCD₃) δ ppm 8.99 (1H, s.), 8.52 (1H, s), 8.17 (1H, s), 7.62 (1H, br. s.), 7.32-7.09 (9H, m), 4.92 (2H, s).

### Example 32

### (5-{3-[5-Chloro-2-(Phenylmethoxy)Phenyl]-2-Thienyl-3-Pyridyl)Methan-1-ol (2a)

To a solution of the ethyl ester of example 31 (8.18 g, 18.2 mmol) in a 1:1 mixture of methanol/THF (50 ml/50 ml) is added NaBH₄ (6.87 g, 182 mmol) then the mixture refluxed overnight to produce the corresponding aldehyde. The reaction is cooled, quenched with HCl 10%, diluted with AcOEt, washed with NaHCO₃, brine, dried (anh. MgSO₄) and concentrated under reduced pressure to give a residue which was purified by flash chromatography (70% CH₂CL₂/30% AcOEt) to provide the title compound (2.96 g). ¹H nmr (400 MHz, CD₃COCD₃) δ ppm 8.45 (1H, d, J=2.0 Hz), 8.32 (1H, d, J=2.3 Hz), 7.68 (1H, d, J=2.2 Hz), 7.53 (1H, d, J=5.2 Hz), 7.29-7.04 (9H, m), 4.89 (3H, s), 4.60 (2H, s).

### Example 33

### 2-(5-{3-[5-Chloro-2-(Phenylmethoxy)Phenyl]-2-Thienyl}-3-Pyridyl)Propan-2-ol (3a)

To a solution of the ethyl ester of example 31 (34 mg, 0.084 mmol) in ether (1.5 mL) at −78° C. was added MeMgBr (3M in ether, 0.11 mL,0.33 mmol) and the reaction was warmed to room temperature and stirred for 1 h. The mixture was quenched with saturated NH₄Cl, diluted with Et₂O and washed successively with HCl 10%, aq. NaHCO₃ and brine. The ether layer was dried with anh. MgSO₄, filtered and concentrated under reduced pressure to give a residue which was purified by flash chromatography (75% Hexanes/25% AcOEt) to provide the title compound (25.6 mg). ¹H nmr (400 MHz, CD₃COCD₃) δ ppm 8.62 (1H, s.), 8.31 (1H, s), 7.66 (1H, s), 7.59 (1H, d, J=7.5 Hz), 7.32-7.11 (9H, m), 4.96 (2H, s), 4.30 (1H, s), 1.39 (6H, s). Elemental analysis calculated for C₂₅H₂₃Cl₂NSO₂: C, 63.56; H, 4.91; N, 2.96; S, 6.79; found: C, 64.47, H, 5.17; N, 3.06; S, 6.58.

### Example 34

### 1-(5-{3-[5-Chloro-2-(Phenylmethoxy)Phenyl](2-Thienyl)}(3-Pyridyl))-2,2,2-Trifluoroethan-1-ol (4a)

To a solution of the aldehyde of example 32 (1.00 g, 2.46 mmol) in THF (10 ml) at 0° C. was added CF₃TMS (0.44 mL, 3.0 mmol) and TBAF (0.25 ml, 1.0M in THF). The reaction was stirred for 2 h. at room temperature and quenched by adding HCl 10% . The mixture was then stirred for 1 h., diluted with AcOEt, washed with HCl 10%, NaHCO₃ (aq), brine, dried (anh. MgSO₄) and concentrated under reduced pressure to give a residue which was purified by flash chromatography (85% CH₂Cl₂/15% AcOEt) to provide the title compound (0.90 g). ¹H nmr (400 MHz, CD₃COCD₃) δ ppm 8.62 (1H, d, J=1.5 Hz), 8.48 (1F[, d, J=2.2 Hz), 7.80 (1H, s), 7.59 (1H, d, J=5.2 Hz), 7.32-7.08 (9H, m), 6.37 (1H, d, J=5.6 Hz), 5.29 (1H, q, J=6.8 Hz), 4.92 (2H, s). Elemental analysis calculated for C₂₄H₁₈NSF₃Cl₂O₂: C, 56.26; H, 3.54; N, 2.73; S, 6.26: found: C, 56.83; H, 3.64; N, 2.80; S, 6.27.

### Example 35

### 1-(5-{3-[5-Chloro-2-(Phenylmethoxy)Phenyl](2-Thienyl)}(3-Pyridyl))-2,2,2-Trifluoroethan-1-One (5a)

To a solution of the alcohol of example 34 (0.7 g, 1.5 mmol) in dichloromethane was added manganese dioxide (1.26×14.5 mmol) and the slurry was stirred overnight at room temperature. The reaction mixture was then filtered through a pad of celite and the volatils were evaporated under reduced pressure to give a residue which was purified by flash chromatography (70% CH₂Cl₂/30% AcOEt) to provide the title compound (0.33 g). ¹H nmr (400 MHz, acetone-d₆) δ ppm 8.78 (1H, d, J=2.1 Hz), 8.48 (1H, d, J=2.2 Hz), 7.96 (1H, t, J=2.1 Hz), 7.60 (1H, d, J=5.1 Hz), 7.35-7.05 (9H, m), 4.94 (2H, s).

What is claimed is:

1. A method of treating or preventing urinary incontinence comprising administering to a mammalian patient in need such treatment or prevention a compound represented by formula I: 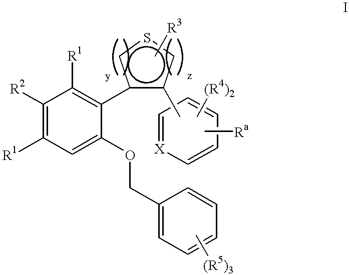
or a pharmaceutically acceptable salt, hydrate or ester thereof, wherein:
X represents C or N;
y and z are independently 0-2, such that y+z=2;
R^{a} is selected from the group consisting of:
1) heteroaryl, wherein heteroaryl is selected from the group consisting of:
a) furyl,
b) diazinyl, triazinyl or tetrazinyl,
c) imidazolyl,
d) isoxazolyl,
e) isothiazolyl,
f) oxadiazolyl,
g) oxazolyl,
h) pyrazolyl,
i) pyrrolyl,
j) thiadiazloyl,
k) thiazolyl
l) thienyl
m) triazolyl and
n) tetrazolyl, wherein heteroaryl is optionally substituted with 1-3 substituents independently selected from R¹¹ or C₁₋₄alkyl,
2) —COR⁶,
3) —NR⁷R⁸,
4) —SO₂R⁹,
5) hydroxy,
6) C₁₋₆alkoxy, optionally substituted with 1-3 substituents independently selected from R¹¹, and
7) C₁₋₆alkyl, C₂₋₆alkenyl or C₃₋₆cycloalkyl, optionally substituted with 1-3 substituents independently selected from R¹¹, and further substituted with 1-3 substituents independently selected from the group consisting of:
(a) —COR⁶
(b) —NR⁷R⁸,
(c) —SO₂R⁹,
(d) hydroxy,
(e) C₁₋₆alkoxy or haloC₁₋₆alkoxy, and
(f) heteroaryl,
such that R^{a} is positioned on the pyridyl ring to which it is bonded in a 1,3 or 1,4 relationship relative to the thienyl group represented in formula I;
R¹, R², R³, R⁴ and R⁵ are independently selected from the group consisting of:
1) hydrogen,
2) halogen,
3) C₁₋₆alkyl,
4) C₁₋₆alkoxy,
5) C₁₋₆alkylthio,
6) nitro,
7) carboxy and
8) CN, wherein items (3)-(5) above are optionally substituted with 1-3 substituents independently selected from R¹¹;
R⁶ is selected from the group consisting of hydrogen, hydroxy, C₁₋₆alkyl, C₁₋₆alkoxy and NR⁷R⁸, wherein C₁₋₆alkyl or C₁₋₆alkoxy are optionally substituted with 1-3 substituents independently selected from R¹¹;
R⁷ and R⁸ are independently selected from the group consisting of:
(1) hydrogen,
(2) hydroxy,
(3) SO₂R⁹
(4) C₁₋₆alkyl,
(5) C₁₋₆alkoxy,
(6) phenyl,
(7) naphthyl,
(8) furyl,
(9) thienyl and
(10) pyridyl, wherein items (4)-(5) above are optionally substituted with 1-3 substituents independently selected from R¹¹, and items (6)-(10) above are optionally substituted with 1-3 substituents independently selected from R¹¹ or C₁₋₄alkyl,
R⁹ is selected from the group consisting of
(1) hydroxy,
(2) N(R¹⁰)₂,
(3) C₁₋₆alkyl, optionally substituted with 1-3 substituents independently selected from R¹¹,
(4) phenyl,
(5) naphthyl,
(6) furyl,
(7) thienyl and
(8) pyridyl, wherein items (4)-(8) above are optionally substituted with 1-3 substituents independently selected from R¹¹ or C₁₋₄alkyl;
R¹⁰ is hydrogen or C₁₋₆alkyl; and
R¹¹ is selected from the group consisting of: halogen, hydroxy, C₁₋₃alkoxy, nitro, N(R¹⁰)₂ and pyridyl
in an amount that is effective for treating or preventing urinary incontinence.

2. A method of treating or preventing incontinence in accordance with claim 1 wherein X represents C.

3. A method of treating or preventing incontinence in accordance with claim 1 wherein X represents N.

4. A method of treating or preventing incontinence in accordance with claim 1 wherein R^{a} is selected from the group consisting of: heteroaryl, COR⁶, C₁₋₆ alkyl and C₂₋₆ alkenyl, optionally substituted as originally defined, and SO₂R⁹.

5. A method of treating or preventing incontinence in accordance with claim 4 wherein R^{a} is selected from the group consisting of:

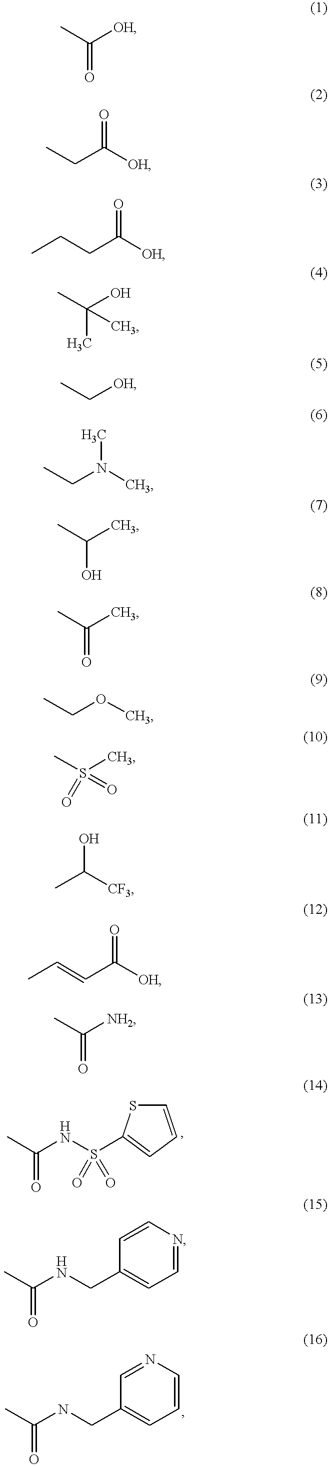

6. A method of treating or preventing incontinence in accordance with claim 1 wherein R^{a} is selected from the group consisting of: CO₂H, CH₂OH, C(OH)(CH₃)₂, CH(OH)CF₃ and C(O)CF₃.

7. A method of treating or preventing incontinence in accordance with claim 1 wherein R¹ and R² are selected from the group consisting of halo, C₁-4 alkyl, C₁₋₄ alkoxy, C₁₋₄ alkylthio and NO₂.

8. A method of treating or preventing incontinence in accordance with claim 1 wherein each R⁴ and R⁵ independently represents a member selected from the group consisting of: H, halo, C₁₋₆ alkyl and C₁₋₆ alkoxy, said alkyl and alkoxy groups being optionally substituted as originally defined.

9. A method of treating or preventing incontinence in accordance with claim 1 wherein each R³ independently represents a member selected from the group consisting of: H and halo.

10. A method of treating or preventing incontinence in accordance with claim 1 wherein one of y and z represents 0 and the other represents 2.

11. A method of treating or preventing incontinence in accordance with claim 1 wherein:
X is C or N;
R^{a} is selected from the group consisting of: heteroaryl, as originally defined, COR⁶, wherein R⁶ is as originally defined, C₁₋₆ alkyl and C₂₋₆ alkenyl, optionally substituted as originally defined, and SO₂R⁹ with R⁹ as originally defined;
R¹ and R² are selected from the group consisting of halo, C₁₋₄ alkyl, C₁₋₄ alkoxy, C₁₋₄ alkylthio and NO₂;
R⁴ and R⁵ independently represent members selected from the group consisting of: H, halo, C₁₋₆ alkyl and C₁₋₆ alkoxy, said alkyl and alkoxy groups being optionally substituted as originally defined;
each R³ independently represents a member selected from the group consisting of: H and halo;
and one of y and z represents 0 and the other represents 2.

12. A method of treating or preventing incontinence in accordance with claim 11 wherein:
X is C or N;
R^{a} is selected from the group consisting of: 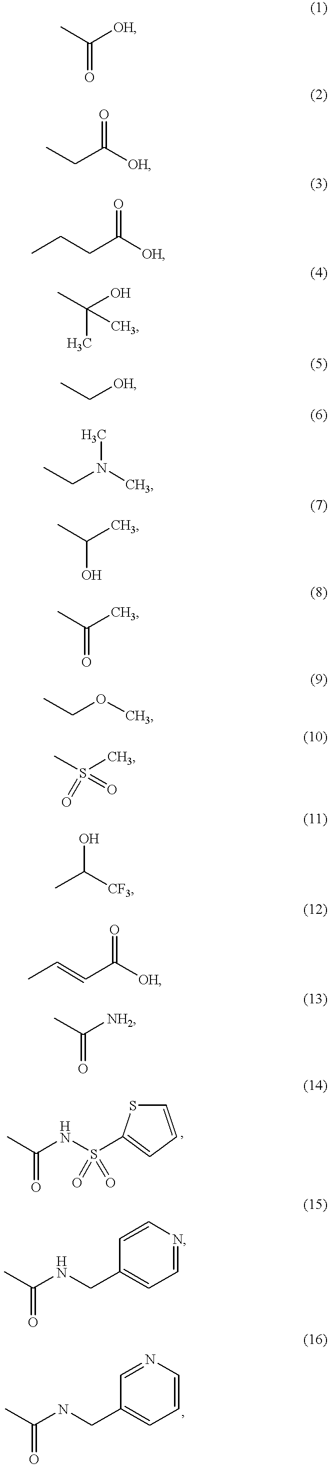
R¹ and R² are selected from the group consisting of halo, C₁₋₄ alkyl, C₁₋₄ alkoxy, C₁₋₄ alkylthio and NO₂;
R⁴ and R⁵ independently represent members selected from the group consisting of: H., halo, C₁₋₆ alkyl and C₁₋₆ alkoxy, said alkyl and alkoxy groups being optionally substituted as originally defined;
each R³ independently represents a member selected from the group consisting of: H and halo;
and one of y and z represents 0 and the other represents 2.

13. A method of treating or preventing incontinence in accordance with claim 1 wherein::
X is C or N;
R^{a} is selected from the group consisting of: CO₂H, CH₂OH, C(OH)(CH₃)₂, CH(OH)CF₃ and C(O)CF₃;
R¹ and R² are selected from the group consisting of halo, C₁₋₄ alkyl, C₁₋₄ alkoxy, C₁₋₄ alkylthio and NO₂;
R⁴ and R⁵ independently represent members selected from the group consisting of: H, halo, C₁₋₆ alkyl and C₁₋₆ alkoxy, said alkyl and alkoxy groups being optionally substituted as originally defined;
each R³ independently represents a member selected from the group consisting of: H and halo;
and one of y and z represents 0 and the other represents 2.

14. A method of treating or preventing incontinence in accordance with claim 1 wherein the compound administered is selected from Table 1 below:
TABLE 1

| COMPOUND | EXAMPLE |
|---|---|
| 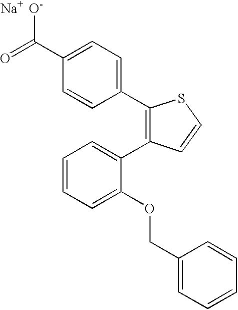 | 1 |
| 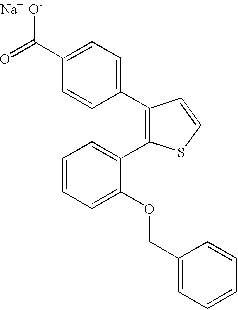 | 2 |
| 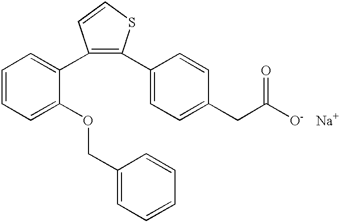 | 3 |
| 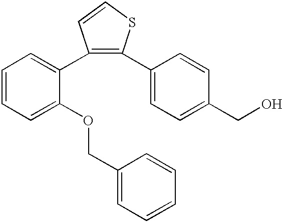 | 4 |
| 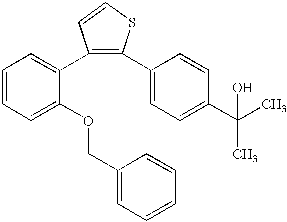 | 5 |
| 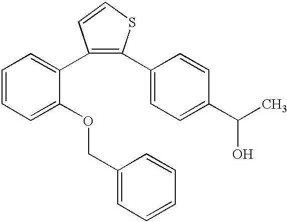 | 6 |
| 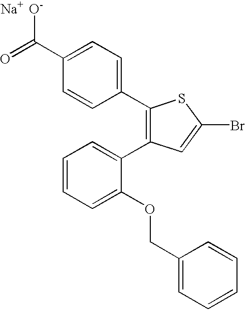 | 7 |
| 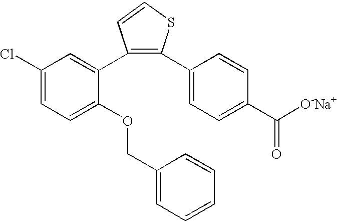 | 8 |
| 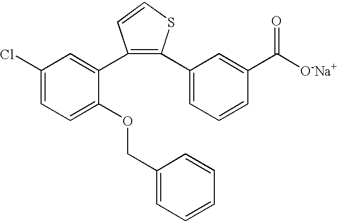 | 9 |
| 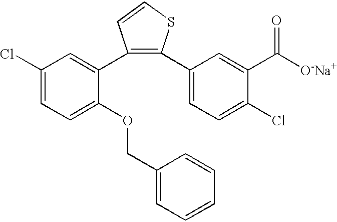 | 10 |
| 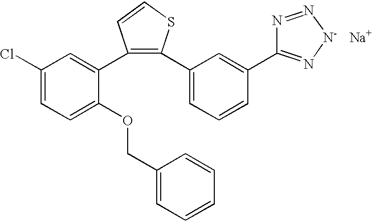 | 11 |
| 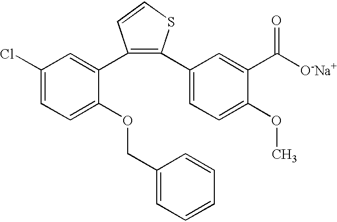 | 12 |
| 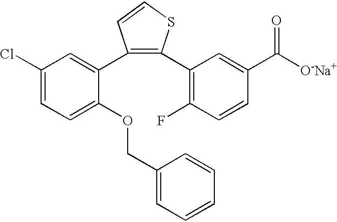 | 13 |
| 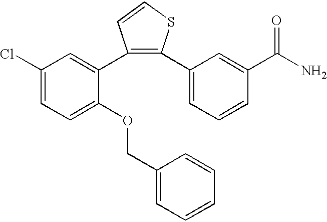 | 14 |
| 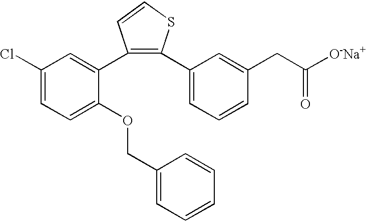 | 15 |
| 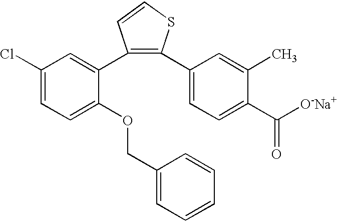 | 16 |
| 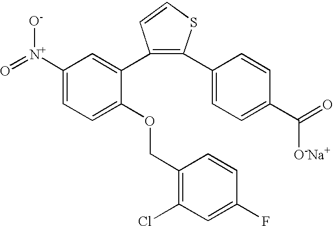 | 17 |
| 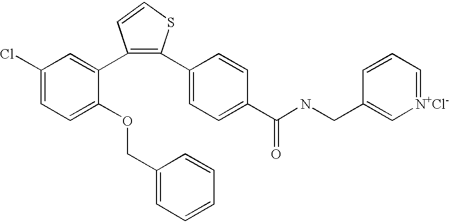 | 18 |
| 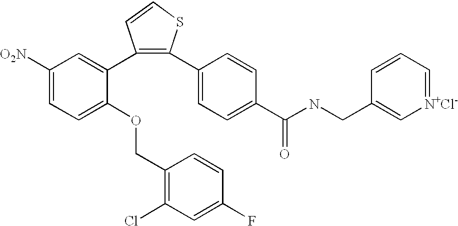 | 19 |
| 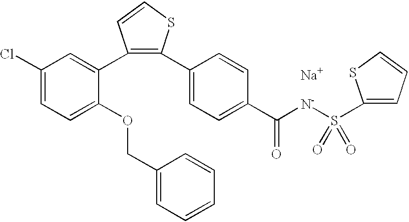 | 20 |
| 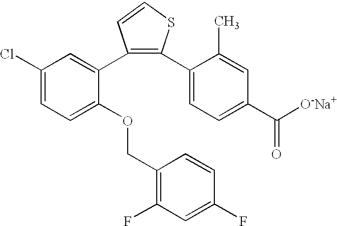 | 21 |
| 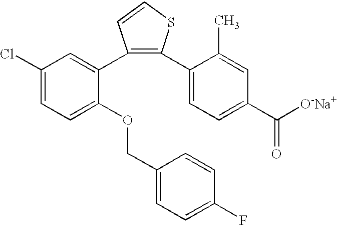 | 22 |
| 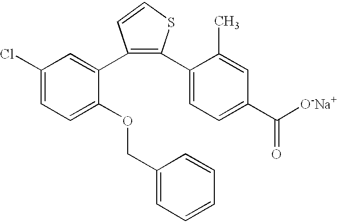 | 23 |
| 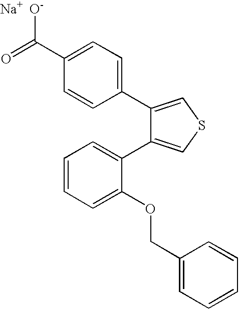 | 25 |
| 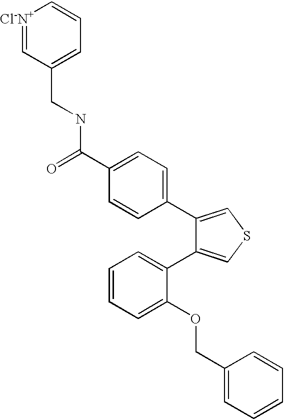 | 26 |
| 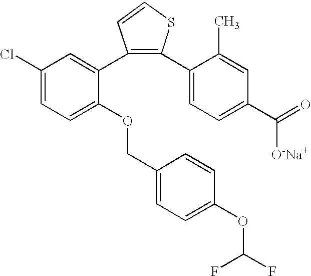 | 27 |
| 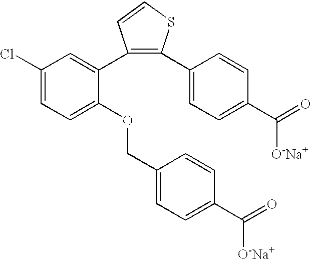 | 28 |
| 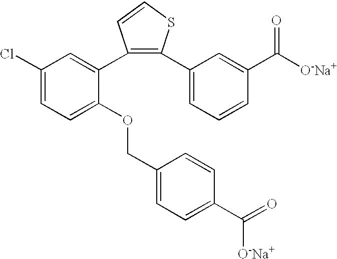 | 29 |
| 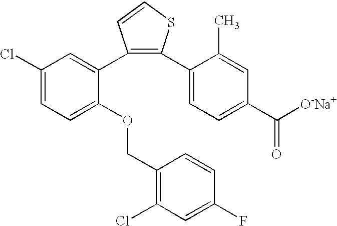 | 30 |
| 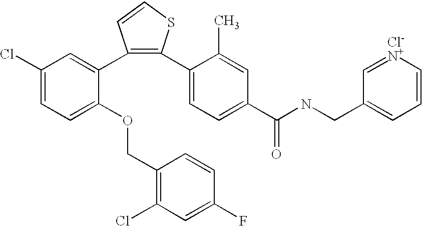 | 31 |
| 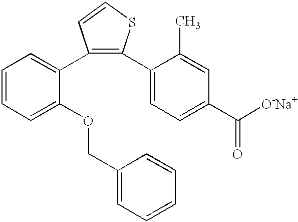 | 37 |
| 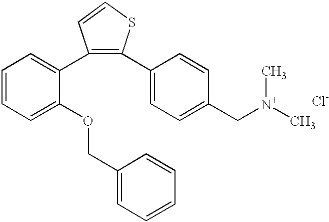 | 38 |
| 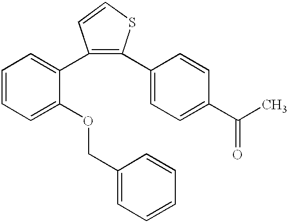 | 39 |
| 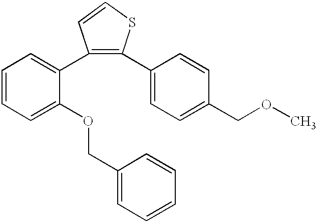 | 40 |
| 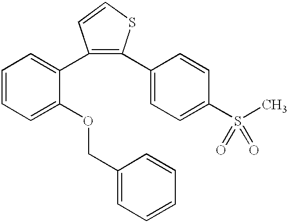 | 41 |
| 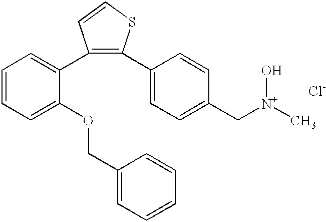 | 43 |
| 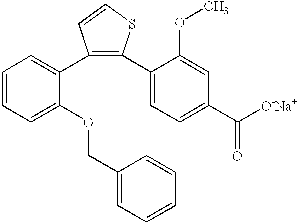 | 44 |
| 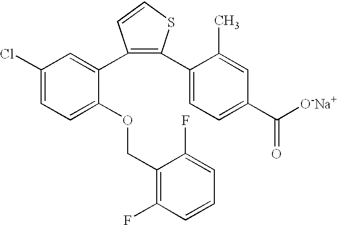 | 45 |
| 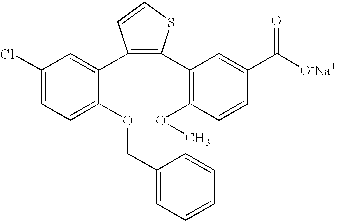 | 46 |
| 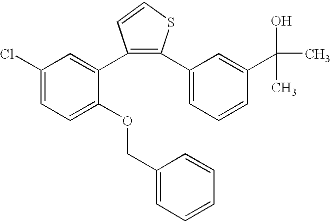 | 47 |
| 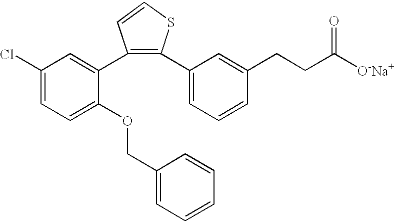 | 48 |
| 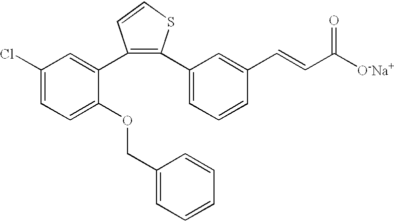 | 49 |
| 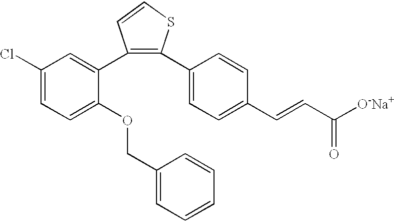 | 50 |
| 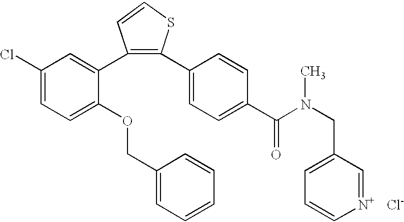 | 52 |
| 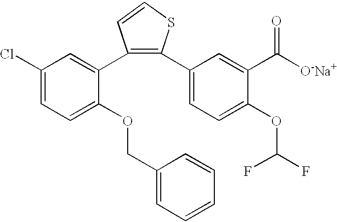 | 53 |
| 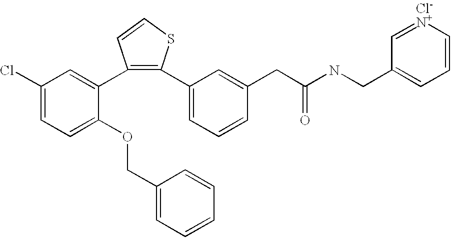 | 55 |
| 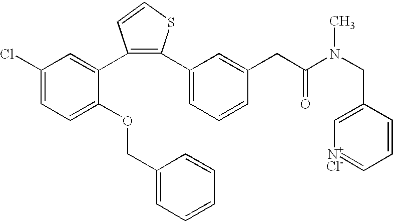 | 56 |
| 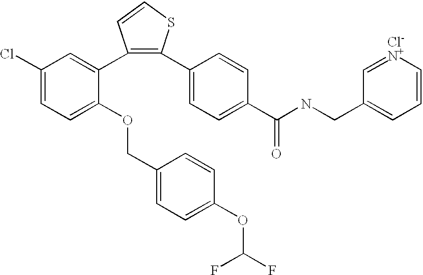 | 57 |
| 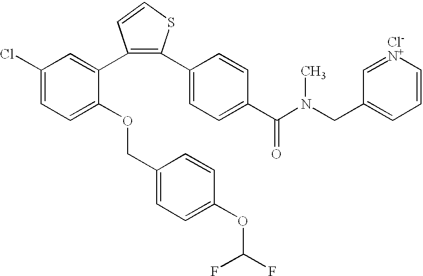 | 58 |
| 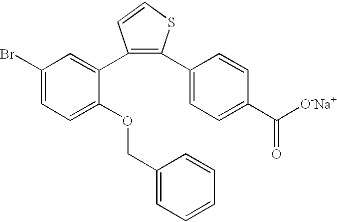 | 59 |
| 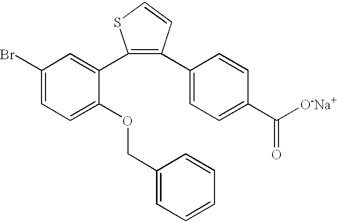 | 60 |
| 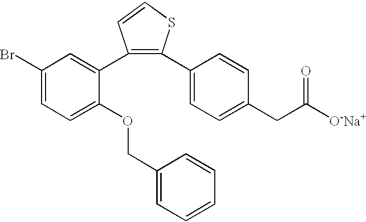 | 61 |
| 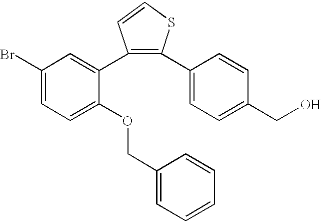 | 62 |
| 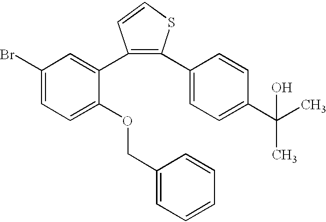 | 63 |
| 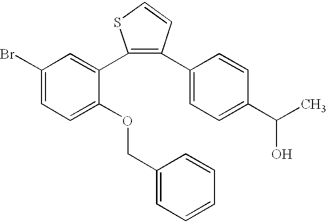 | 64 |
| 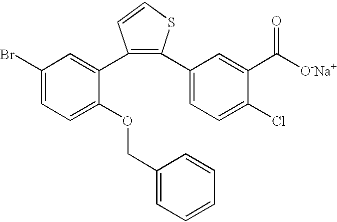 | 65 |
| 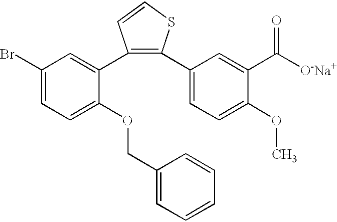 | 66 |
| 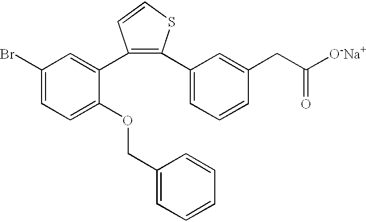 | 67 |
| 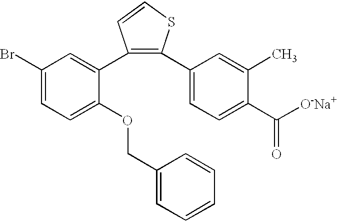 | 68 |
| 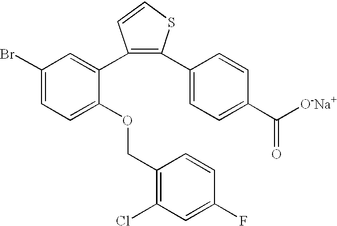 | 69 |
| 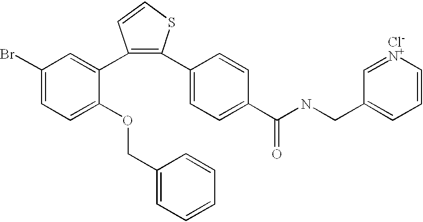 | 70 |
| 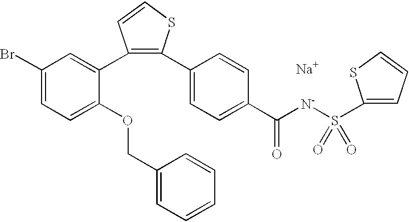 | 71 |
| 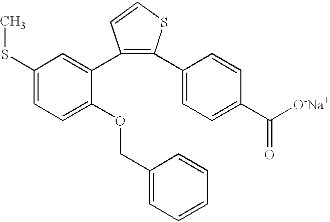 | 76 |
| 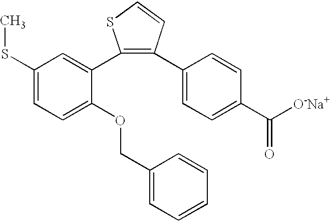 | 77 |
| 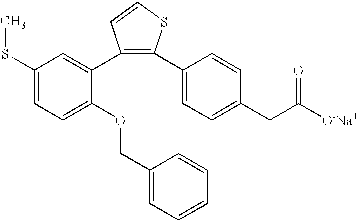 | 78 |
| 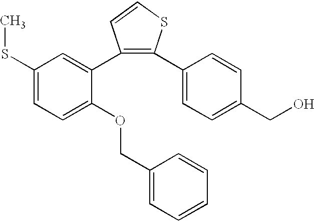 | 79 |
| 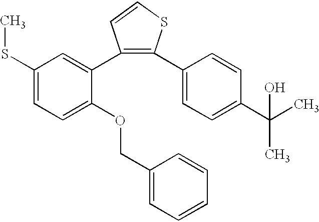 | 80 |
| 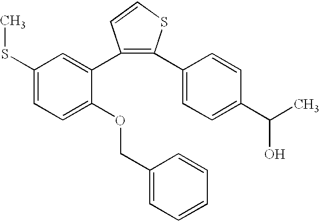 | 81 |
| 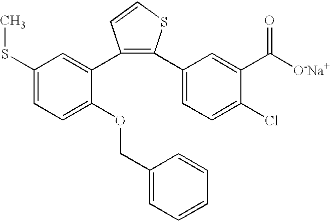 | 82 |
| 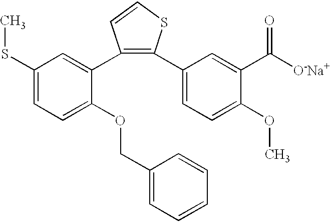 | 83 |
| 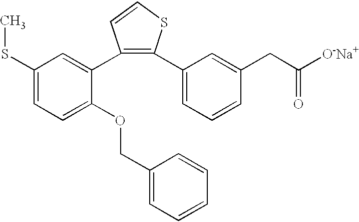 | 84 |
| 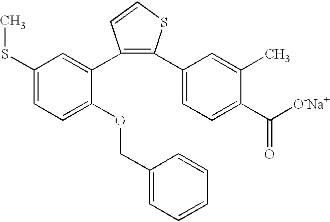 | 85 |
| 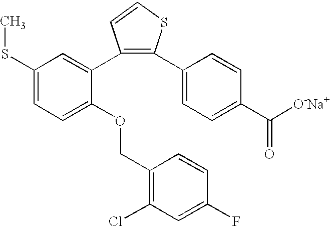 | 86 |
| 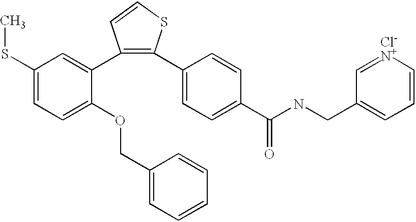 | 87 |
| 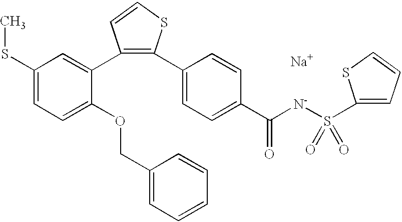 | 88 |

TABLE 1

| COMPOUND | EXAMPLE |
|---|---|
| 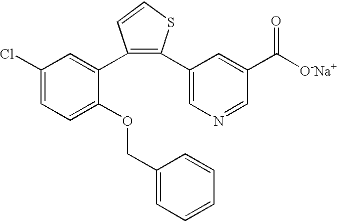 | 1a |
| 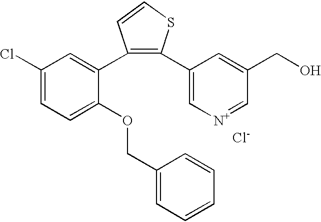 | 2a |
| 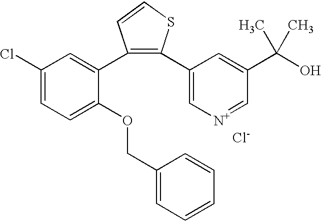 | 3a |
| 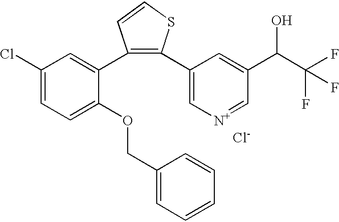 | 4a |
| 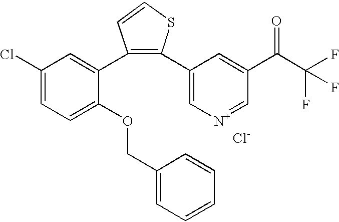 | 5a |
| 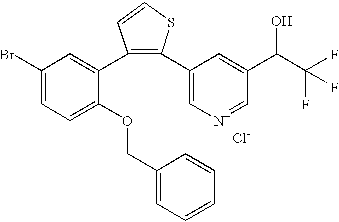 | 6a |
| 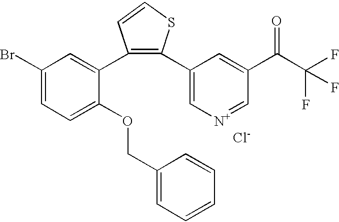 | 7a |

or a pharmaceutically acceptable salt or hydrate thereof.

15. A method of treating or preventing incontinence in accordance with claim 1 wherein the compound of formula I is co-administered with another agent selected from the group consisting of: a cyclooxygenase-2 selective inhibiting drug, a diuretic, a potassium supplement, a potassium channel modulator, a urinary antiseptic, a muscle relaxant, an alpha adrenoceptor antagonist, a beta adrenoceptor agonist, an anticholinergic, a calcium channel antagonist, an antimuscarinic, an estrogen replacement, a neurokinin receptor antagonist, a dopamine receptor ligand, a phosphodiesterase inhibitor and an antidepressant.

16. A method of treating or preventing incontinence in accordance with claim 1 wherein the compound of formula I is co-administered with another agent selected from the group consisting of: baclofen and capsaicin.

17. A pharmaceutical composition comprised of a compound of formula I in combination with at least one member selected from the group consisting of: a cyclooxygenase-2 selective inhibiting drug, a diuretic, a potassium supplement, a potassium channel modulator, a urinary antiseptic, a muscle relaxant, an alpha adrenoceptor antagonist, a beta adrenoceptor agonist, an anticholinergic, a calcium channel antagonist, an antimuscarinic, an estrogen replacement, a neurokinin receptor antagonist, a dopamine receptor ligand, a phosphodiesterase inhibitor and an antidepressant, in combination with a pharmaceutically acceptable carrier.

18. A pharmaceutical composition comprising a compound of formula I and another agent selected from the group consisting of: baclofen and capsaicin in combination with a pharmaceutically acceptable carrier.
